(19) United States
(12) Patent Application Publication
(10) Pub. No.: US 2017/0184613 A1
(43) Pub. Date: Jun. 29, 2017

(54) EXOSOME AND LIPID BIOMARKERS FOR MEMORY LOSS

(71) Applicants: GEORGETOWN UNIVERSITY, Washington, DC (US); UNIVERSITY OF ROCHESTER, Rochester, NY (US)

(72) Inventors: Massimo S. FIANDACA, Millersville, MD (US); Mark E. MAPSTONE, Pittsford, NY (US); Howard J. FEDEROFF, Bethesda, MD (US)

(21) Appl. No.: 15/312,804

(22) PCT Filed: May 26, 2015
(86) PCT No.: PCT/US15/32490

§ 371 Date: Nov. 21, 2016

Related U.S. Application Data
(60) Provisional application No. 62/002,453, filed on May 23, 2014.

(51) Int. Cl. G01N 33/68; G01N 33/92

(52) U.S. Cl. CPC G01N 33/6896; G01N 33/92; G01N 2800/2814

(57) ABSTRACT

The present invention relates to methods of determining if a subject has an increased risk of suffering from memory impairment. The methods comprise analyzing at least one sample from the subject to determine a value of the subject's exosomal profile or combined biomarker profile (lipids plus exosomal cargo) and comparing the value of the subject's exosomal or combined biomarker profile with the value of a normal exosomal or biomarker profile, respectively. A change in the value of the subject's exosomal or combined biomarker profile, including a change in the subject's exosomal or combined biomarker profile, over normal values is indicative that the subject has an increased risk of suffering from memory impairment compared to a normal individual.

### STATEMENT REGARDING FEDERALLY SPONSORED RESEARCH OR DEVELOPMENT

Part of the work performed during development of this invention utilized U.S. Government funds under National Instituted of Health Grant No. R01 AG030753 and Department of Defense Contract No. W81XWH-09-1-0107. The U.S. Government has certain rights in this invention.

### BACKGROUND OF THE INVENTION

Field of the Invention

The present invention relates to methods of determining if a subject has an increased risk of suffering from memory impairment. The methods comprise analyzing at least one sample from the subject to determine a value of at least the subject's exosomal profile and comparing the value of the subject's exosomal profile with the value of a normal exosomal profile. A change in the value of the subject's exosomal profile, including a change in the subject's exosomal profile, over normal values is indicative that the subject has an increased risk of suffering from memory impairment compared to a normal individual.

Background of the Invention

Alzheimer's disease (AD) is a neurodegenerative disorder characterized by a progressive dementia that insidiously and inexorably robs older adults of their memory and other cognitive abilities. The prevalence of AD is expected to double every 20 years from 35.6 million individuals worldwide in 2010 to 115 million affected individuals by 2050. There is no cure and current therapies are unable to slow the disease progression.

Early detection of the at-risk population (preclinical), or those in the initial symptomatic stages (prodromal) of AD, may present opportunities for more successful therapeutic intervention, or even disease prevention by interdicting the neuropathological cascade that is ultimately characterized by the deposition of extracellular β-amyloid (Aβ), the most common pathologic being Aβ₁₋₄₂, and accumulation of intracellular neurofibrillary tangles (NFTs) of hyperphosphorylated microtubule associated protein tau (MAPT) within the brain. Tau levels are typically quantified and expressed as total tau and the various phosphorylated tau (p-Tau) species. Multiple p-Tau species have been defined. Six predominant tau structural isoforms, produced by alternate splicing, exist in adult human brains. The proline rich region, midway between the N- and C-terminals of the molecule, is extensively phosphorylated in AD. Two of the most common p-Tau species quantified in AD studies include p-Tau-t181 (phosphorylated at tyrosine 181) and p-Tau-s396 (phosphorylated at serine 396). Aβ₁₋₄₂ and p-Tau species are known to show dysregulated levels within the cerebrospinal fluid (CSF) and brain of prodromal and manifest AD subjects. While p-Tau-t181 is typically altered during the early to mid stages of the evolving AD neuropathology, p-Tau-s396 becomes overexpressed during the later stages of disease and is accumulated within NFTs. Specific AD-related P-tau species, however, have only been discovered within the nervous system, or in rare cases within the skeletal muscle of patients with sporadic inclusion body myositis (sIBM). The expression of AD-related p-Tau species, therefore, is commonly indicative of a nervous system origin, or neural derivation, of the specific tau protein(s).

Exosomes are lipid bilayer nanocontainers (typically 50-100 nm in diameter), released from all viable cells by a membrane fusion process involving the late endosome/multivesicular bodies (MVB) and the plasma membrane. Exosomal cargos are sorted and enriched via a complex mechanism using specific lipids and enriched membrane protein species found within intracellular endosomal structures. Exosomes are formed from specialized portions of these enriched endosomal membranes that invaginate within the endosomal structure to form intraluminal vesicles (ILVs), allowing the endosomal structure to be renamed a MVB. Once released from the cell, exosomes convey certain cytosolic proteins and nucleic acids, in addition to unique quantities of membrane lipids and proteins, and provide a unique form of intercellular communication. Upon fusion of the MVB with the cell membrane, the contained ILVs are released from the cell as exosomes, freely diffusing within the extracellular fluid (ECF). All cells within the nervous system are known to produce exosomes.

Biomarkers for early AD, including CSF tau and Aβ levels, structural and functional magnetic resonance imaging (MRI), and the recent use of brain positron emission tomography (PET) amyloid imaging, are of limited use as widespread screening tools since they provide diagnostic options that are either invasive (i.e., require lumbar puncture), time-consuming (i.e., several hours in a scanner for most comprehensive imaging protocols), or expensive. No current blood-based biomarkers can detect incipient dementia with the required sensitivity and specificity during the preclinical stages. Continued interest in blood-based biomarkers remains because these specimens are obtained using minimally invasive, rapid, and relatively inexpensive methods. With recent technological advances in ‘omics’ technologies and systems biology analytic approaches, the comprehensive bioinformatic analyses of blood-based biomarkers may not only yield improved accuracy in predicting those at risk, but may also provide new insights into the underlying mechanisms and pathobiological networks involved in AD and possibly herald the development of new therapeutic strategies.

The preclinical interval resulting in prodromal (mild cognitive impairment (MCI)) or manifest AD is known to be variable, multifactorial, and extends for at least 7-10 years prior to the emergence of clinical signs. In the absence of accurate and easily obtained biomarkers, multimodal neurocognitive testing remains the most accurate, standardized, and widely used pre-mortem screening method to determine the presence or absence of clinical MCI or AD. The utility of strict cognitive assessment for preclinical stages of MCI or AD is limited, however, as this approach is not only time-consuming but is expected, by definition, to be normal in cognitively normal preclinical subjects. Neuropsychological testing is able to quantitatively delineate specific brain alterations from normal, such as deficiencies in memory, attention, language, visuoperceptual, and executive functions, which are typically not known to be affected in individuals during the preclinical stages. Thus, information obtained from multiple diagnostic studies will probably be most useful in defining the MCI/AD preclinical stages, including neuropsychological testing and some form(s) of biomarker(s). While CSF and neuroimaging have been used to define clinical MCI/AD to date, their clinical utility as screening tools for asymptomatic preclinical individuals is not established.

### SUMMARY OF THE INVENTION

The present invention relates to methods of determining if a subject has an increased risk of suffering from memory impairment. The methods comprise analyzing at least one specimen, a plasma sample for example, from the subject to determine a value of the subject's exosomal profile and comparing the value of the subject's exosomal profile with the value of a normal exosomal profile. A change in the value of the subject's exosomal profile, including a change in the subject's exosomal cargo protein profile, above (or possibly below) normal values is indicative that the subject has an increased risk of suffering from memory impairment compared to a normal individual.

In other embodiments, the methods also comprise analyzing at least one sample from the subject to determine a value of the subject's exosomal profile and lipid profile and comparing the value of the subject's combined biomarker profile (lipidomic profile plus exosomal profile) with the value of a normal biomarker profile. In other embodiments, the methods also comprise analyzing at least one sample from the subject to determine a value of the subject's biomarker profile, with the biomarker profile comprising constituents of an exosomal profile and a lipid profile, and comparing the subject's biomarker profile with the value of a normal biomarker profile. A change in the value of the subject's biomarker profile, however calculated, over normal values is indicative that the subject has an increased risk of suffering from memory impairment compared to a normal individual.

### BRIEF DESCRIPTION OF THE DRAWINGS

FIG. 1 depicts the quantitative differences in specific AD-related cargo proteins from neurally-derived plasma exosomes. Box/whisker (and outlier) representations of ELISA results provide evidence for significant difference (p<0.001) for each of four exosome cargo protein levels between the Normal Control (NC) group and the other cognitively unimpaired group (Converterₚᵣₑ), and with the clinically symptomatic Converterₚₒₛₜ and aMCI/AD groups.

FIG. 2 depicts the Receiver Operating Characteristic (ROC) curves indicating differentiation of Normals from Converterₚᵣₑ utilizing each of four individual exosome cargo proteins. (a) Total tau provides an AUC of 98.5% (96.4%-100%), while (b) pTau-t181 provides an AUC of 100% (100%-100%), (c) pTau-s396 gives an AUC of 97.4% (93.2%-100%), and (d) Ab1-42 provides an AUC of 100% (100%-100%). Shaded areas on the ROC curve depict the 95% confidence intervals (also in parentheses after AUC).

FIG. 3 depicts Receiver Operating Characteristic (ROC) curve and the Plasma Exosome Index (PEI) box plot for four combined neurally-derived plasma exosome cargo proteins. (a) ROC curve allows differentiation of Converterₚᵣₑ from NC utilizing four combined exosome cargo proteins in a single classifier. (b) Box plots of the Plasma Exosome Index (PEI) results based on the logistic regression model using four exosome cargo proteins to distinguish NC and the Converterₚᵣₑ groups. Despite the higher variability in the latter group, there is no overlap between the two plots.

FIG. 4 depicts the Receiver Operating Characteristic (ROC) curves allowing differentiation of Normals from MCI/AD utilizing each of four exosome cargo proteins. (a) Total tau provides an AUC of 100%, while (b) pTau-t181 provides an AUC of 100%, (c) pTau-s396 gives an AUC of 100%, and (d) Aβ₁₋₄₂ provides an AUC of 100%.

FIG. 5 depicts the ROC curves allowing differentiation of Normals from Converterₚₒₛₜ utilizing each of four exosome cargo proteins. (a) Total tau provides an AUC of 98.1%, while (b) pTau-t181 provides an AUC of 100%, (c) pTau-s396 gives an AUC of 98.9%, and (d) Aβ₁₋₄₂ provides an AUC of 100%.

FIG. 6 depicts ROC curves allowing differentiation of MCI/AD from Converterₚᵣₑ utilizing each of four exosome cargo proteins. (a) Total tau provides an AUC of 59.1%, while (b) pTau-t181 provides an AUC of 66.5%, (c) pTau-s396 gives an AUC of 100%, and (d) Aβ₁₋₄₂ provides an AUC of 62.8%.

FIG. 7 depicts ROC curves allowing differentiation of MCI/AD from Converterₚₒₛₜ utilizing each of four exosome cargo proteins. (a) Total tau provides an AUC of 63.5%, while (b) pTau-t181 provides an AUC of 59.4%, (c) pTau-s396 gives an AUC of 85.6%, and (d) Aβ₁₋₄₂ provides an AUC of 62.0%.

FIG. 8 depicts ROC curves allowing differentiation of Converterₚᵣₑ from Converterₚₒₛₜ utilizing each of four exosome cargo proteins. (a) Total tau provides an AUC of 55.8%, while (b) pTau-t181 provides an AUC of 72.8%, (c) pTau-s396 gives an AUC of 68.1%, and (d) Aβ₁₋₄₂ provides an AUC of 49.8%.

### DETAILED DESCRIPTION OF THE INVENTION

The present invention relates to methods of determining if a subject has an increased risk of suffering from memory impairment. The methods comprise analyzing at least one sample from the subject to determine a value of at least the subject's exosomal profile and comparing the value of the subject's exosomal profile with the value of a normal exosomal profile. A change in the value of the subject's exosomal profile, including a change in the subject's exosomal profile, over normal values is indicative that the subject has an increased risk of suffering from memory impairment compared to a normal individual.

In additional embodiments, the methods comprise analyzing at least one plasma sample from the subject to determine a value of the subject's lipidomic profile, and also analyzing the exosomal profile and comparing the value of the subject's biomarker profile (lipidomic profile plus exosomal profile) with the value of a normal biomarker profile. In other embodiments, the methods also comprise analyzing at least one sample from the subject to determine a value of the subject's biomarker profile, with the biomarker profile comprising constituents of an exosomal profile and a lipid profile, and comparing the subject's biomarker profile with the value of a normal biomarker profile. A change in the value of the subject's biomarker profile compared to normal values is indicative that the subject has an increased risk of suffering from memory impairment compared to a normal individual.

As used herein, the term subject or “test subject” indicates a mammal, in particular a human or non-human primate. The test subject may or may not be in need of an assessment of a predisposition to memory impairment. For example, the test subject may have a condition or may have been exposed to injuries or conditions that are associated with memory impairment prior to applying the methods of the present invention. In another embodiment, the test subject has not been identified as a subject that may have a condition or may have been exposed to injuries or conditions that are associated with memory impairment prior to applying the methods of the present invention.

As used herein, the phrase “memory impairment” means a measureable or perceivable decline or decrease in the subject's ability to recall past events. As used herein, the term “past events” includes both recent (new) events (short-term memory) or events further back in time (long-term memory). In one embodiment, the methods are used to assess an increased risk of short-term memory impairment. In another embodiment, the methods are used to assess an increased risk in long-term memory impairment. The memory impairment can be age-related memory impairment. The memory impairment may also be disease-related memory impairment. Examples of disease-related memory impairment include but are not limited to Alzheimer's Disease, Parkinson's Disease, Multiple Sclerosis, Huntington's Disease, Pick's Disease, Progressive Supranuclear Palsy, Brain Tumor(s), Head Trauma, and Lyme Disease to name a few. In one embodiment, the memory impairment is related to amnestic mild cognitive impairment (aMCI). In another embodiment, the memory impairment is related to Alzheimer's Disease. The root cause of the memory impairment is not necessarily critical to the methods of the present invention. The measureable or perceivable decline in the subject's ability to recall past events may be assessed clinically by a health care provider, such as a physician, physician's assistant, nurse, nurse practitioner, psychologist, psychiatrist, hospice provider, or any other provider that can assess a subject's memory. The measureable or perceivable decline in the subject's ability to recall past events may be assessed in a less formal, non-clinical manner, including but not limited to the subject himself or herself, acquaintances of the subject, employers of the subject and the like. The invention is not limited to a specific manner in which the subject's ability to recall past events is assessed. In fact, the methods of the invention can be implemented without the need to assess a subject's ability to recall past events. Of course, the methods of the present invention may also include assessing the subject's ability to assess past events one or more times, before determining the subject's exosomal profile after determining the subject's exosomal profile at least one time.

In one embodiment, the decline or decrease in the ability to recall past events is relative to each individual's ability to recall past events prior to the diagnosed decrease or decline in the ability to recall past events. In another embodiment, the decline or decrease in the ability to recall past events is relative to a population's (general, specific or stratified) ability to recall past events prior to the diagnosed decrease or decline in the ability to recall past events.

As used herein, the term means “increased risk” is used to mean that the test subject has an increased chance of developing or acquiring memory impairment compared to a normal individual. The increased risk may be relative or absolute and may be expressed qualitatively or quantitatively. For example, an increased risk may be expressed as simply determining the subject's exosomal profile or biomarker profile and placing the patient in an “increased risk” category, based upon previous population studies. Alternatively, a numerical expression of the subject's increased risk may be determined based upon the exosomal profile or biomarker profile. As used herein, examples of expressions of an increased risk include but are not limited to, odds, probability, odds ratio, p-values, attributable risk, relative frequency, positive predictive value, negative predictive value, and relative risk.

For example, the correlation between a subject's exosomal profile and the likelihood of suffering from memory impairment may be measured by an odds ratio (OR) and by the relative risk (RR). Similarly, the correlation between a subject's biomarker profile and the likelihood of suffering from memory impairment may be measured by an odds ratio (OR) and by the relative risk (RR). If P(R⁺) is the probability of developing memory impairment for individuals with the risk profile (R) and P(R⁻) is the probability of developing memory impairment for individuals without the risk profile, then the relative risk is the ratio of the two probabilities: RR=P(R⁺)/P(R⁻).

In case-control studies, however, direct measures of the relative risk often cannot be obtained because of sampling design. The odds ratio allows for an approximation of the relative risk for low-incidence diseases and can be calculated: OR=(F⁺/(1−F⁺))/(F⁻/(1−F⁻)), where F⁺ is the frequency of a exosomal risk profile in cases studies and F⁻ is the frequency of exosomal risk profile (or biomarker risk profile) in controls. F⁺ and F⁻ can be calculated using the exosomal profile or biomarker profile frequencies of the study.

The attributable risk (AR) can also be used to express an increased risk. The AR describes the proportion of individuals in a population exhibiting memory impairment due to one or more specific members of an exosomal profile or biomarker profile. AR may also be important in quantifying the role of individual components (specific members) in disease etiology and in terms of the public health impact of the individual marker. The public health relevance of the AR measurement lies in estimating the proportion of cases of memory impairment in the population that could be prevented if the profile or individual component were absent. AR may be determined as follows: AR=P_{E}(RR−1)/(P_{E}(RR−1)+1), where AR is the risk attributable to a profile or individual component of the profile, and P_{E} is the frequency of the profile or individual component of the profile within the population at large. RR is the relative risk, which can be approximated with the odds ratio when the profile or individual component of the profile under study has a relatively low incidence in the general population.

In one embodiment, the increased risk of a patient can be determined from p-values that are derived from association studies. Specifically, associations with specific profiles can be performed using regression analysis by regressing the exosomal profile and/or biomarker profile with memory impairment. In addition, the regression may or may not be corrected or adjusted for one or more factors. The factors for which the analyses may be adjusted include, but are not limited to age, sex, weight, ethnicity, geographic location, fasting state, state of pregnancy or post-pregnancy, menstrual cycle, general health of the subject, alcohol or drug consumption, caffeine or nicotine intake and circadian rhythms, and the subject's apolipoprotein epsilon (APOE) genotype to name a few.

Increased risk can also be determined from p-values that are derived using logistic regression. Binomial (or binary) logistic regression is a form of regression that is used when the dependent is a dichotomy and the independents are of any type. Logistic regression can be used to predict a dependent variable on the basis of continuous and/or categorical independents and to determine the percent of variance in the dependent variable explained by the independents; to rank the relative importance of independents; to assess interaction effects; and to understand the impact of covariate control variables. Logistic regression applies maximum likelihood estimation after transforming the dependent into a “logit” variable (the natural log of the odds of the dependent occurring or not). In this way, logistic regression estimates the probability of a certain event occurring. These analyses can be conducted with the program SAS.

SAS (“statistical analysis software”) is a general-purpose package (similar to Stata and SPSS) created by Jim Goodnight and N.C. State University colleagues. Ready-to-use procedures handle a wide range of statistical analyses, including but not limited to, analysis of variance, regression, categorical data analysis, multivariate analysis, survival analysis, psychometric analysis, cluster analysis, and nonparametric analysis.

As used herein, the phrase “exosomal profile” means a collection of one or more measurements, such as but not limited to a quantity or concentration, for individual molecules taken from exosomes that exist in test sample taken from the subject. Examples of test samples or sources of test samples for the exosomal profile include, but are not limited to, biological fluids, which can be tested by the methods of the present invention described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

As used herein, the phrase “lipidomic profile” or “lipid profile” means a collection of measurements, such as but not limited to a quantity or concentration, for individual lipids taken from a test sample of the subject. As used herein, a lipidomic profile is not generated from the lipid component of the exosome or exosomal cargo, but is generated from the non-exosomal lipids isolated from the test samples. Examples of test samples or sources of components for the lipidomic profile include, but are not limited to, biological fluids, which can be tested by the methods of the present invention described herein, and include but are not limited to whole blood, such as but not limited to peripheral blood, serum, plasma, cerebrospinal fluid, urine, amniotic fluid, lymph fluids, and various external secretions of the respiratory, intestinal and genitourinary tracts, tears, saliva, milk, white blood cells, myelomas and the like. Test samples to be assayed also include but are not limited to tissue specimens including normal and abnormal tissue.

As used herein, the phrase “biomarker profile” or “combined profile” or “combined biomarker profile” means either the combination of a subject's lipidomic profile and the subject's exosomal profile, i.e., an exosome profile and a lipid profile are calculated separately and then combined, or biomarker profile can be created by creating a single profile using with at least one lipid member used to generate the lipidomic profile and at least member used to generate the exosomal profile. For example, one example of a “biomarker profile” could be generated by measuring one member from the exosomal profile, e.g., total Tau, and at least one member of the lipidomic profile, e.g., propionyl AC (pAC). In short, a “biomarker profile” a used herein requires at least one exosomal component and at least one lipid component, whereas an “exosome profile” is comprised purely of exosomal components as defined herein and a “lipid profile” is comprised purely of lipid components as defined herein.

Techniques to assay levels of individual components of the lipidomic profile from test samples are well known to the skilled technician, and the invention is not limited by the means by which the components are assessed. In one embodiment, levels of the individual components of the lipidomic profile are assessed using mass spectrometry in conjuncton with ultra-performance liquid chromatography (UPLC), high-performance liquid chromatography (HPLC), and UPLC to name a few. Other methods of assessing levels of the individual components include biological methods, such as but not limited to ELISA assays.

The assessment of the levels of the individual components of the lipidomic profile can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the lipidomic profile, a sample is taken from the subject. The sample may or may not processed prior assaying levels of the components of the lipidomic profile. For example, whole blood may be taken from an individual and the blood sample may be processed, e.g., centrifuged, to isolate plasma or serum from the blood. The sample may or may not be stored, e.g., frozen, prior to processing or analysis.

Individual components of the lipidomic profile include but are not limited to phosphatidyl cholines (PC) lyso PCs and acylcarnitines (AC). Specific examples of PCs, lyso PCs and ACs that can be included as constituents of the lipidomic profile include but are not limited to (1) propionyl AC (pAC), (2) lyso PC a C18:2, (3) PC aa C36:6, (4) C16:1-OH (Hydroxyhexadecenoyl-L-carnitine), (5) PC aa C38:0, (6) PC aa 36:6, (7) PC aa C40:1, (8) PC aa C40:2, (9) PC aa C40:6 and (10) PC ae C40:6. Those of skill in the art will recognize the specific identity of each constituent listed based upon the nomenclature above. For example, lipds (5) (PC aa C38:0) is known to those of skill in the art as phosphatidylcholine diacyl C 38:0, lipid (10) (PC ae C40:6) is known as phosphatidylcholine acyl-alkyl C 40:6 and lipid (2) (lyso PC a C18:2) is known as lysoPhosphatidylcholine acyl C18:2. In one embodiment, the individual levels of each of the lipids are lower than those compared to normal levels. In another embodiment, one, two, three, four, five, six, seven, eight or nine of the levels of each of the lipids are lower over normal levels.

The levels of depletion of the lipids over normal levels can vary. In one embodiment, the levels of (1) propionyl AC (pAC) are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (2) lyso PC a C18:2 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (3) PC aa C36:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (4) C16:1-OH (Hydroxyhexadecenoyl-L-carnitine), are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (5) PC aa C38:0 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (6) PC aa 36:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (7) PC aa C40:1 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (8) PC aa C40:2 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (9) PC aa C40:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. In one embodiment, the levels of (10) PC ae C40:6 are at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 lower than normal levels. For the purposes of the present invention, the number of “times” the levels of a lipid is lower or higher over normal can be a relative or absolute number of times. In the alternative, the levels of the lipids may be normalized to a standard and these normalized levels can then be compared to one another to determine if a lipid is lower or higher.

For the purposes of the present invention the lipidomic profile comprises at least two, three, four, five, six, seven, eight, nine or all ten lipids listed above. If two lipids are used in generating the lipidomic profile, any combination of two of 1-10 listed above can be used. If three lipids are used in generating the lipidomic profile, any combination of three of 1-10 listed above can be used. If four lipids are used in generating the lipidomic profile, any combination of four of 1-10 listed above can be used. If five lipids are used in generating the lipidomic profile, any combination of five of 1-10 listed above can be used. If six lipids are used in generating the lipidomic profile, any combination of six of 1-10 listed above can be used. If seven lipids are used in generating the lipidomic profile, any combination of seven of 1-10 listed above can be used. If eight lipids are used in generating the lipidomic profile, any combination of eight of 1-10 listed above can be used. If nine lipids are used in generating the lipidomic profile, any combination of nine of 1-10 listed above can be used. Of course, all ten lipids of 1-10 above can be used to generate the lipidomic profile.

In one embodiment, the test sample for the exosomal profile and/or the lipidomic profile is taken from the subject's blood. The blood can be processed to isolate components such as the cellular component, plasma and serum. In one embodiment, the test sample is whole blood. In another embodiment, the test sample is serum. In another embodiment, the test sample is plasma.

Regardless of the source of the test sample, the test sample can also be processed to isolate or enrich the sample for neurally derived exosomes. As used herein, the term “neurally derived exosome” is used to mean an exosome that displays or contains, i.e., the exosomes are “positive for,” one or more neural cell markers, i.e., exosomes that contain markers indicating that they derived from the nervous system. Thus, the neural cell markers can be markers of any cell type typically associated with the nervous system, such as it but not limited to, neurons, astrocytes, oligodendrocytes, and microglia to name a few. See Noble, M., et al., Nature, 316(6030):725-728 (1985), which is incorporated by reference. Examples of neural cell markers include but are not limited to neuronal cell adhesion molecule (NCAM, also known in the art as CD56), nerve growth factor receptor (NGFR), L1 neural cell adhesion molecule, ephrin A2, ephrin A4, ephrin A5, ephrin B1, ephrin B2, GAP-43, Laminin-1, NAP-22, Netrin-1, neutropilin, plexin-A1, semaphorin 3A, semaphorin 3F, semaphorin 4D, Trk A, LINGO-1, GAD65, neural cell surface antigen (A2B5) to name a few. The neural cell markers may, but need not, be markers normally present on the cell surface of neural cells. The invention is not limited to the specific neural markers on the surface or within the exosomes or the methods used to isolate these “neurally derived exosomes.”

In one embodiment, the neurally derived exosomes used to generate the exosomal profile display or contain NCAM, i.e., “NCAM positive.” In another embodiment, the neurally derived exosomes display or contain display at least one of NCAM, nerve growth factor receptor (NGFR), L1 neural cell adhesion molecule, ephrin A2, ephrin A4, ephrin A5, ephrin B1, ephrin B2, GAP-43, Laminin-1, NAP-22, Netrin-1, neutropilin, plexin-A1, semaphorin 3A, semaphorin 3F, semaphorin 4D, Trk A, LINGO-1, GAD65. The invention is not limited to the number of markers on the neurally-derived exosomes.

Thus “neurally enriched exosomes” or “enriched for neurally derived exosomes” are phrases used to indicate that the sample has been enriched for exosomes displaying or containing neurally derived exosomes. The enrichment need not be 100%, such that a small fraction of non-neurally derived exosomes can be present in the enriched sample. In one specific embodiment, the population of exosomes used for analysis in the present application has been enriched to at least 50% neurally derived exosomes. In other specific embodiments, the population of exosomes used for analysis in the present application has been enriched to at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% neurally derived exosomes. In one specific embodiment, the population of exosomes used for analysis in the present application has been enriched to 100% neurally derived exosomes.

Techniques to enrich exosomes that display or contain a particular marker, for example a neural cell marker, are well known in the art. For example, techniques involving immunoisolation of exosomes using antibodies are well-known. See Lasser, C. et al., J. Visualized Experiments, (59), e3037, doi: 10.3791/3037 (2012), which is incorporated by reference, for techniques involving isolation of RNA from exosomes. Other techniques for isolating exosomes include but are not limited to precipitation techniques, chromatography, ultracentrifugation and immunosorbent beads.

Once the exosomes are isolated, the contents of the exosomes, the “exosome cargo” can be analyzed for the presence or absence of specific molecules. The exosome cargo includes molecules embedded in the surface of the phospholipid bilayer membrane of the exosomal vesicles as well as molecules contained within the contents of the exosomal vesicles. The exosome cargo includes but is not limited to, species of lipids, RNA, DNA, proteins, peptides and other metabolites, including but not limited to carbohydrates. Each of these classes of molecules can be considered to be a “component” or “constituent” of the exosomal profile, e.g., the lipidomic component within the exosomal profile, the protein component within the exosomal profile, etc. To be clear, the lipidomic profile, as used herein, is not the same as the lipid component within the exosomal profile. In other words, the lipidomic profile is generated from lipids found in the plasma, but not within exosomes. In another embodiment, the exosomal cargo used in the analytic methods of the present invention includes an RNA component. In this specific embodiment, the RNA may be micro RNA (miRNA), messenger RNA (mRNA), ribosomal RNA (rRNA), other non-coding RNA (ncRNA), or any type of RNA. Well known in the art, miRNA is generally considered to be an ncRNA and non-rRNA containing about 30 or fewer bases. Unless specified otherwise, the term miRNA is used herein to include any RNA that is about 30 bases or shorter in length, including but not limited to ncRNA, coding RNA, non-rRNA and rRNA. Subsets of miRNA can be can be used in the methods of the present invention include but are not limited to ncRNA, coding RNA, non-rRNA and rRNA. In another embodiment, the exosomal cargo used in the analytic methods of the present invention includes a protein component, such as a species of phospholipase or lysophopholipase. The RNA components typically reside within the exosomal vesicle. The proteins examined in the exosomal cargo may or may not be whole proteins, or fragments thereof. The protein components may exist within the exosomal bilayer membrane compartment and/or within the exosomal vesicle.

Once the exosomal cargo has been isolated, the identity of the components can be ascertained and their amounts, levels, concentrations, quantities, etc. can be assessed. Techniques to assay levels of individual components of the exosomal cargo from test samples are well known to the skilled technician, and the invention is not limited by the means by which the components are assessed. Membrane components can be separated from the components within the exosomal vesicle for separate analyses. In one embodiment, levels of the individual components of the exosomal cargo are assessed using, PCR, quantitative PCR, Western blot, Northern blot, Southern blot, ELISA assays, mass spectrometry in conjunction with ultra-performance liquid chromatography (MS-UPLC), high-performance liquid chromatography (HPLC), and UPLC to name a few. The methods of assessing the individual components of the exosomal cargo will depend on the type of molecule to be assessed, i.e., protein or peptide levels may be assessed by different methods than for assessing lipid levels.

The assessment of the levels of the individual components of the exosomal cargo can be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the individual components of the exosomal cargo, a sample is taken from the subject. The sample may or may not processed prior assaying levels of the components of the exosomal cargo. For example, whole blood may be taken from an individual and the blood sample may be processed, e.g., centrifuged, to isolate plasma or serum from the blood. The sample may or may not be stored, e.g., frozen, prior to processing or analysis.

Techniques to assay levels of individual protein components of the exosomal profile from test samples are well known to the skilled technician, and the invention is not limited by the means by which the components are assessed. In one embodiment, levels of the protein components of the exosomal profile are assessed using quantitative arrays, ELISA, Western Blot analysis, mass spectroscopy, high-performance liquid chromatography (HPLC) and the like. To determine levels of proteins, it is not necessary that an entire protein be present or fully sequenced. In other words, determining levels of, for example, a fragment of a protein being analyzed may be sufficient to conclude or assess that the individual is present or absent. Similarly, if, for example, arrays or blots are used to determine protein levels, the presence/absence/strength of a detectable signal will be sufficient to assess protein levels without the need isolate and/or determine the full length protein.

The assessment of the levels of the protein components of the exosomal profile can also be expressed as absolute or relative values and may or may not be expressed in relation to another component, a standard an internal standard or another molecule of compound known to be in the sample. If the levels are assessed as relative to a standard or internal standard, the standard may be added to the test sample prior to, during or after sample processing.

To assess levels of the protein components of the exosomal profile, a sample is taken from the subject. The sample may or may not processed prior assaying levels of the components of the exosomal profile. For example, whole blood may be taken from an individual and the blood sample may be processed, e.g., centrifuged, to isolate specific cells, e.g., leukocytes, from the blood. The sample may or may not be stored, e.g., frozen, prior to processing or analysis.

Individual protein components of exosomal profile include but are not limited to (A) amyloid beta 1-42 protein (Aβ₁₋₄₂), (B) total Tau protein (tT), (C) phosphorylated Tau at T181 (pT181) and (D) phosphorylated Tau at 5396 (pS396). A large number of additional exosomal proteins have been identified (on the internet at exocarta.org/#), and include, but are not limited to, various chaperone and enzymatic protein species, including kinases, phosphatases, phospholipases and lysophospholipases. In one embodiment, the protein levels in the exosomal cargo are increased compared to levels found in exosomes from normal subjects. In another embodiment, one, two, three or four of the proteins are increased over normal levels.

The increased protein in the exosomal cargo over normal levels can vary. In one embodiment, the levels of (A) Aβ₁₋₄₂ are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of (B) total Tau (tT) protein are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of (C) tau-T181 (pT181) are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of (D) tau-S396 (pS396) are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of (E) phospholipase A2 (pA2) are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels. In one embodiment, the levels of (F) a phosphatase or kinase are increased at least 1.05, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20 times or more over normal levels.

For the purposes of the present invention the exosomal profile comprises at least two, three, four, five or six proteins or fragments thereof. If two proteins are used in generating the exosomal profile, any combination of two of A-F listed above can be used. If three proteins are used in generating the exosomal profile, any combination of three of A-F listed above can be used. If four proteins are used in generating the exosomal profile, any combination of three of A-F listed above can be used. If five proteins are used in generating the exosomal profile, any combination of three of A-F listed above can be used. If six proteins are used in generating the exosomal profile, all of A-F listed above can be used. In specific embodiments, the exosomal profile comprises or consists of levels of Aβ₁₋₄₂. In other embodiments, the exosomal profile comprises or consists of levels of total Tau. In another embodiment, the exosomal profile comprises or consists of levels of pT181. In another embodiment, the exosomal profile comprises or consists of levels of pS396. In another embodiment, the exosomal profile comprises or consists of levels of Aβ₁₋₄₂ and levels and total Tau, comprises or consists of levels of Aβ₁₋₄₂ and levels and pT181, or comprises or consists of levels of Aβ₁₋₄₂ and levels and pS396. In another embodiment, the exosomal profile comprises or consists of levels of total Tau and levels and pS396, or comprises or consists of levels of total tau and levels and pT181. In another embodiment, the exosomal profile comprises or consists of levels of pT181 and levels and pS396. In another embodiment, the exosomal profile comprises or consists of levels of Aβ₁₋₄₂, levels of total Tau and levels pT181, comprises or consists of levels of Aβ₁₋₄₂, levels of total Tau and levels of pS396, comprises or consists of levels of levels of Aβ₁₋₄₂, levels of pS396 and levels pT181, or comprises or consists of levels of total Tau, levels of tau-T181 and levels of tau-S396. In another embodiment, the exosomal profile comprises and consists of levels of Aβ₁₋₄₂, levels of pS396, levels pT181 and levels of total Tau. Various combinations of constituents used to generate exosomal profiles (or biomarker profiles) are outlined in Table 1 below. As used herein, “levels” is not limited to a specific measurement, such as absolute concentration, ratio, etc., but is intended to be used as a general term that can mean any quantitative measurement of a components, such as but not limited to absolute concentration, relative concentration, percent, ratio, log ratio, relative amount, absolute amount and the like.

In one embodiment, the exosomal profile is assessed prior to determination of the lipidomic profile. In another embodiment, the lipidomic profile is assessed prior to the determination of the exosomal profile. In another embodiment, exosomal component(s) and the lipidomic component(s) of the biomarker profile are assessed at the same time or during the same assay.

In select embodiments, the subject's exosomal profile is compared to the profile that is deemed to be a normal exosomal profile. To establish the exosomal profile of a normal individual, an individual or group of individuals may be first assessed for their ability to recall past events to establish that the individual or group of individuals has a normal or acceptable ability memory. Once established, the exosomal profile of the individual or group of individuals can then be determined to establish a “normal exosomal profile.” In one embodiment, a normal exosomal profile can be ascertained from the same subject when the subject is deemed to possess normal cognitive abilities and no signs (clinical or otherwise) of memory impairment. In one embodiment, a “normal” exosomal profile is assessed in the same subject from whom the sample is taken prior to the onset of measureable, perceivable or diagnosed memory impairment. That is, the term “normal” with respect to an exosomal profile can be used to mean the subject's baseline exosomal profile prior to the onset of memory impairment. The exosomal profile can then be reassessed periodically and compared to the subject's baseline exosomal profile. Thus, the present invention also include methods of monitoring the progression of memory impairment in a subject, with the methods comprising determining the subject's exosomal profile more than once, i.e., at least a first a second time point, over a period of time. For example, some embodiments of the methods of the present invention will comprise determining the subject's exosomal profile two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having memory impairment would also include embodiments in which the subject's profile is assessed during and after treatment of memory impairment. In other words, the present invention also includes methods of monitoring the efficacy of treatment of memory impairment by assessing the subject's exosomal profile over the course of the treatment and after the treatment. The treatment may be any treatment designed to increase a subject's ability to recall past events, i.e., improve a subject's memory.

In other embodiments, a normal exosomal profile is assessed in a sample from a different subject or patient (from the subject being analyzed) and this different subject does not have or is not suspected of having memory impairment. In still another embodiment, the normal exosomal profile is assessed in a population of healthy individuals, the constituents of which display no memory impairment. Thus, the subject's exosomal profile can be compared to a normal exosomal profile generated from a single normal sample or an exosomal profile generated from more than one normal sample.

In select embodiments, the subject's combined biomarker profile is compared to the profile that is deemed to be a normal combined biomarker profile. To establish the combined biomarker profile of a normal individual, an individual or group of individuals may be first assessed for their ability to recall past events to establish that the individual or group of individuals has a normal or acceptable ability memory. Once established, the combined biomarker profile of the individual or group of individuals can then be determined to establish a “normal combined biomarker profile” (or “normal biomarker profile” or “normal combined profile”). In one embodiment, a normal combined biomarker profile can be ascertained from the same subject when the subject is deemed to possess normal cognitive abilities and no signs (clinical or otherwise) of memory impairment. In one embodiment, a “normal combined biomarker” profile is assessed in the same subject from whom the sample is taken prior to the onset of measureable, perceivable or diagnosed memory impairment. That is, the term “normal” with respect to a combined biomarker profile can be used to mean the subject's baseline combined biomarker profile prior to the onset of memory impairment. The combined biomarker profile can then be reassessed periodically and compared to the subject's baseline combined biomarker profile. Thus, the present invention also include methods of monitoring the progression of memory impairment in a subject, with the methods comprising determining the subject's combined biomarker profile more than once, i.e., at least a first a second time point, over a period of time. For example, some embodiments of the methods of the present invention will comprise determining the subject's combined biomarker profile two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having memory impairment would also include embodiments in which the subject's profile is assessed during and after treatment of memory impairment. In other words, the present invention also includes methods of monitoring the efficacy of treatment of memory impairment by assessing the subject's combined biomarker profile over the course of the treatment and after the treatment. The treatment may be any treatment designed to increase a subject's ability to recall past events, i.e., improve a subject's memory.

In other embodiments, a normal combined biomarker profile is assessed in a sample from a different subject or patient (from the subject being analyzed) and this different subject does not have or is not suspected of having memory impairment. In still another embodiment, the normal combined biomarker profile is assessed in a population of healthy individuals, the constituents of which display no memory impairment. Thus, the subject's combined biomarker profile can be compared to a normal combined biomarker profile generated from a single normal sample or a combined biomarker profile generated from more than one normal sample.

The table below lists various non-limiting embodiments for the components to be used in generating the various combined biomarker profiles that can be used in the methods of the present invention. Any combination of levels of exosomal cargo proteins can be combined with one or up to all of the lipid components of the lipidomic profile disclosed herein to create a combined biomarker profile. of course, if no lipid constituents are used, then the profile would be considered an exosomal profile. Likewise, if no exosomal constituents are used, then the profile would be considered a lipid profile. As discussed herein, a purely exosomal profile can be combined with a purely lipidomic profile to generate a combined biomarker profile, and individual constituents used to generate each profile can be used to generate a single profile.

TABLE 1

| | | lyso PC | PC aa | C16:1- | PC aa | PC aa | PC aa | PC aa | PC aa | PC ae |
|---|---|---|---|---|---|---|---|---|---|---|
| | pAC | a C18:2 | C36:6 | OH | C38:0 | 36:6 | C40:1 | C40:2 | C40:6 | C40:6 |
| Aβ₁₋₄₂ | +/− | +/− | +/− | +/− | +/− | +/− | +/− | +/− | +/− | +/− |
| tT | +/− | +/− | +/− | +/− | +/− | +/− | +/− | +/− | +/− | +/− |
| pT181 | +/− | +/− | +/− | +/− | +/− | +/− | +/− | +/− | +/− | +/− |
| pS396 | +/− | +/− | +/− | +/− | +/− | +/− | +/− | +/− | +/− | +/− |
| Aβ₁₋₄₂ + tT | +/− | +/− | +/− | +/− | +/− | +/− | +/− | +/− | +/− | +/− |
| Aβ₁₋₄₂ + pT181 | +/− | +/− | +/− | +/− | +/− | +/− | +/− | +/− | +/− | +/− |
| Aβ₁₋₄₂ + pS396 | +/− | +/− | +/− | +/− | +/− | +/− | +/− | +/− | +/− | +/− |
| tT + pT181 | +/− | +/− | +/− | +/− | +/− | +/− | +/− | +/− | +/− | +/− |
| tT + pS396 | +/− | +/− | +/− | +/− | +/− | +/− | +/− | +/− | +/− | +/− |
| pT181 + pS396 | +/− | +/− | +/− | +/− | +/− | +/− | +/− | +/− | +/− | +/− |
| Aβ₁₋₄₂ + tT + pT181 | +/− | +/− | +/− | +/− | +/− | +/− | +/− | +/− | +/− | +/− |
| Aβ₁₋₄₂ + tT + pS396 | +/− | +/− | +/− | +/− | +/− | +/− | +/− | +/− | +/− | +/− |
| Aβ₁₋₄₂ + pT181 + pS396 | +/− | +/− | +/− | +/− | +/− | +/− | +/− | +/− | +/− | +/− |
| tT + pS396 + pT181 | +/− | +/− | +/− | +/− | +/− | +/− | +/− | +/− | +/− | +/− |
| Aβ₁₋₄₂ + tT + pT181 + pS396 | +/− | +/− | +/− | +/− | +/− | +/− | +/− | +/− | +/− | +/− |

Table 2 shows 15 select embodiments, and, of course, other embodiments besides the 15 listed below are encompassed within Table 1.

TABLE 2

| | | lyso PC | PC aa | C16:1- | PC aa | PC aa | PC aa | PC aa | PC aa | PC ae |
|---|---|---|---|---|---|---|---|---|---|---|
| | pAC | a C18:2 | C36:6 | OH | C38:0 | 36:6 | C40:1 | C40:2 | C40:6 | C40:6 |
| Aβ₁₋₄₂ | + | + | + | + | + | + | + | + | + | + |
| tT | + | + | + | + | + | + | + | + | + | + |
| pT181 | + | + | + | + | + | + | + | + | + | + |
| pS396 | + | + | + | + | + | + | + | + | + | + |
| Aβ₁₋₄₂ + tT | + | + | + | + | + | + | + | + | + | + |
| Aβ₁₋₄₂ + pT181 | + | + | + | + | + | + | + | + | + | + |
| Aβ₁₋₄₂ + pS396 | + | + | + | + | + | + | + | + | + | + |
| tT + pT181 | + | + | + | + | + | + | + | + | + | + |
| tT + pS396 | + | + | + | + | + | + | + | + | + | + |
| pT181 + pS396 | + | + | + | + | + | + | + | + | + | + |
| Aβ₁₋₄₂ + tT + pT181 | + | + | + | + | + | + | + | + | + | + |
| Aβ₁₋₄₂ + tT + pS396 | + | + | + | + | + | + | + | + | + | + |
| Aβ₁₋₄₂ + pT181 + pS396 | + | + | + | + | + | + | + | + | + | + |
| tT + pS396 + pT181 | + | + | + | + | + | + | + | + | + | + |
| Aβ₁₋₄₂ + tT + pT181 + pS396 | + | + | + | + | + | + | + | + | + | + |

To establish the exosomal or biomarker profile of a normal individual, an individual or group of individuals may be first assessed for their ability to recall past events to establish that the individual or group of individuals has a normal or acceptable ability memory. Once established, the exosomal or biomarker profile of the individual or group of individuals can then be determined to establish a “normal exosomal profile” or “normal biomarker profile.” In one embodiment, a normal exosomal or biomarker profile can be ascertained from the same subject when the subject is deemed to possess normal cognitive abilities and exhibit no signs (clinical or otherwise) of memory impairment. In one embodiment, a “normal exosomal profile” or “normal biomarker profile” is assessed in the same subject from whom the sample is taken prior to the onset of measureable, perceivable or diagnosed memory impairment. That is, the term “normal” with respect to an exosomal or biomarker profile can be used to mean the subject's baseline exosomal or biomarker profile prior to the onset of memory impairment. The exosomal or biomarker profile can then be reassessed periodically and compared to the subject's baseline exosomal or biomarker profile. Thus, the present invention also includes methods of monitoring the progression of memory impairment in a subject, with the methods comprising determining the subject's exosomal profile or biomarker profile more than once over a period of time. For example, some embodiments of the methods of the present invention will comprise determining the subject's exosomal profile two, three, four, five, six, seven, eight, nine, 10 or even more times over a period of time, such as a year, two years, three, years, four years, five years, six years, seven years, eight years, nine years or even 10 years or longer. In other embodiments, the methods of the present invention will comprise determining the subject's biomarker profile two, three, four, five, six, seven, eight, nine, 10 or even more times over a period of time, such as a year, two years, three, years, four years, five years, six years, seven years, eight years, nine years or even 10 years or longer. The methods of monitoring a subject's risk of having memory impairment would also include embodiments in which the subject's profile is assessed during and after treatment of memory impairment. In other words, the present invention also includes methods of monitoring the efficacy of treatment of memory impairment by assessing the subject's exosomal or biomarker profile over the course of the treatment and/or after the treatment. The treatment may be any treatment designed to increase a subject's ability to recall past events, i.e., improve a subject's memory.

Of course, measurements of the individual components, e.g., concentration, ratios, levels, etc., of the normal exosomal or biomarker profile can fall within a range of values, and values that do not fall within this “normal range” are said to be outside the normal range. These measurements may or may not be converted to a value, number, factor or score as compared to measurements in the “normal range.” For example, a measurement for a specific protein component within the exosomal cargo, or a specific lipid component of the lipidomic profile that is below the normal range, may be assigned a value or −1, −2, −3, etc., depending on the scoring system devised.

In one embodiment, the “exosomal profile value” can be a single value, number, factor or score given as an overall collective value to the individual molecular components of the profile, or to the categorical components, i.e., the RNA component and the protein component. For example, if each component is assigned a value, such as above, the exosomal value may simply be the overall score of each individual or categorical value. For example, if four components are used to generate the protein component and two of the components are assigned values of “−2” and two are assigned values of “−1,” the protein component of the exosomal profile value in this example would be −6, with a normal value being, for example, “0.” Continuing the example, if three components are used to generate the RNA component and two of the components are assigned values of “2” and one is assigned values of “−1,” the RNA component of the exosomal profile value in this example would be 3, with a normal value being, for example “0.” In this manner, the exosomal profile value could be useful single number or score, the actual value or magnitude of which could be an indication of the actual risk of memory impairment, e.g., the “more negative” or “more positive” the value, the greater the risk of memory impairment. Moreover, if 10 components are used to generate the lipid profile and five of the components are assigned values of “−2” and five are assigned values of “−1,” the value of the lipid profile in this example would be −15, with a normal value being, for example, “0.” Thus, continuing the example from above, the combination of the lipid profile value and the protein component of the exosome profile value (collectively, the biomarker profile value) would be −21.

In another embodiment either the “exosomal profile value” or the “biomarker profile value” can be a series of values, numbers, factors or scores given to the individual components of the overall profile. In another embodiment, the “exosomal profile value” or the “biomarker profile value” may be a combination of values, numbers, factors or scores given to individual components of the profile as well as values, numbers, factors or scores collectively given to a group of components. For example, the measurements of the phosphatidylcholines in the lipid profile may be grouped into one composite score and individual acylcarnitines may be grouped into another composite score. In another example, the exosomal profile value or the biomarker profile value may comprise or consist of individual values, number, factors or scores for specific components, e.g., total Tau, as well as values, numbers, factors or scores for a group on components.

In another embodiment individual values from the components of the exosomal profile or biomarker profile can be used to develop a single score, such as a “exosomal index,” or “biomarker index” which may utilize weighted scores from the individual biomarker values reduced to a diagnostic number value. The combined exosomal or biomarker index may also be generated using non-weighted scores from the individual values from the constituents tested. The exosomal index may also be called a “plasma exosomal index” if the exosomes are harvested from the plasma. The exosomal index may be called a “serum exosomal index” if the exosomes are harvested from serum. The exosomal index may be called a “CSF exosomal index” if the exosomes are harvested from the cerebrospinal fluid. The biomarker index may also be called a “plasma biomarker index” (or “plasma combined biomarker index,” or “plasma combined index”) if the components (exosomes and lipids) are harvested from the plasma. The biomarker index may be called a “serum biomarker index” (or “serum combined biomarker index,” or “serum combined index”) if the components (exosomes and lipids) are harvested from serum. The biomarker index may be called a “CSF biomarker index” (or “CSF combined biomarker index,” or “CSF combined index”) if the components (lipids and exosomes) are harvested from the cerebrospinal fluid. Accordingly, the exosomal or biomarker index can be named after the source of the exosomes or the components of the biomarker profile as a means to further identify the index. When the “exosomal index” or “biomarker index” exceeds (or is less than) a specific threshold level, the individual has a high risk of memory impairment, whereas the maintaining a normal range value of the “exosomal index” or “biomarker index” would indicate a low or minimal risk of memory impairment. In these embodiments, the threshold value would be set by the exosomal index or biomarker index from normal subjects.

In another embodiment, the value of the exosomal profile or biomarker profile can be the collection of data from the individual measurements and need not be converted to a scoring system, such that the “exosomal profile value” or “biomarker profile value” is a collection of the individual measurements of the individual components of the profile. For example, the value of the exosomal component of the combined biomarker profile may be a collection of measurements.

In specific embodiments, a subject is diagnosed of having an increased risk of suffering from memory impairment if six of the subject's protein components of the exosomal profile described herein are at abnormal levels, e.g., all of the protein components of the exosomal cargo are higher than normal levels. In another embodiment, a subject is diagnosed of having an increased risk of suffering from memory impairment if five of the subject's protein components of the exosomal profile described herein are at abnormal levels. In another embodiment, a subject is diagnosed of having an increased risk of suffering from memory impairment if four of the subject's protein components of the exosomal profile described herein are at abnormal levels. In another embodiment, a subject is diagnosed of having an increased risk of suffering from memory impairment if three of the subject's protein components of the exosomal profile described herein are at abnormal levels. In another embodiment, a subject is diagnosed of having an increased risk of suffering from memory impairment if two of the subject's protein components of the exosomal profile described herein are at abnormal levels. In another embodiment, a subject is diagnosed of having an increased risk of suffering from memory impairment if one of the subject's protein components of the exosomal profile described herein is at abnormal levels.

If it is determined that a subject has an increased risk of memory impairment, the attending health care provider may subsequently prescribe or institute a treatment program. In this manner, the present invention also provides for methods of screening individuals as candidates for treatment of memory impairment. The attending healthcare worker may begin treatment, based on the subject's exosomal or combined biomarker profile, before there are perceivable, noticeable or measurable signs of memory impairment in the individual.

Similarly, the invention provides methods of monitoring the effectiveness of a treatment for memory impairment. Once a treatment regimen has been established, with or without the use of the methods of the present invention to assist in a diagnosis of memory impairment, the methods of monitoring a subject's exosomal or combined biomarker profile over time can be used to assess the effectiveness of a memory impairment treatment. Specifically, the subject's exosomal or combined biomarker profile can be assessed over time, including before, during and after treatments for memory impairment. The exosomal or combined biomarker profile can be monitored, with, for example, a decline in the values of the constituents comprising the profile over time, towards the normal values, being indicative that the treatment may be efficacious.

All patents and publications mentioned in this specification are indicative of the level of those skilled in the art to which the invention pertains. All patents and publications cited herein are incorporated by reference to the same extent as if each individual publication was specifically and individually indicated as having been incorporated by reference in its entirety.

### EXAMPLES

### Example 1

Neurocognitive Methods

A total of 525 volunteers participated in this study as part of the Rochester/Orange County Aging Study (R/OCAS), an ongoing natural history study of cognition in community-dwelling older adults. Briefly, participants were followed with yearly cognitive assessments and blood samples were collected following an overnight fast and withholding of all medications. At baseline and each yearly visit, participants completed assessments in such as activities in daily living, memory complaints, signs and symptoms of depression, and were administered a detailed cognitive assessment.

For this study, data from the cognitive tests were used to classify participants into groups for biomarker discovery. Standardized scores (Z-scores) were derived for each participant on each cognitive test and the composite Z-scores were computed for five cognitive domains (attention, executive, language, memory, visuoperceptual) (Table 3).

TABLE 3

| Attention (Zₐₜₜ) | Executive (Zₑₓₑ) | Language (Zᵢₐₙ) | Visuoperceptual (Zᵥᵢₛ) | Memory (Zₘₑₘ) |
|---|---|---|---|---|
| Wechsler | Wechsler | 1-min Category | Hooper Visual | Rey Auditory |
| Memory Scale-III | Memory Scale-III | fluency (Animals) | Organization Test | Verbal Learning |
| Forward Digit | Backward Digit | | (HVOT) | Test Learning |
| Span (WMS-III | Span (WMS-III | | | (RAVLT Learning) |
| FDS) | BDS) | | | |
| Trail Making Test- | Trail Making Test- | Boston Naming | | Rey Auditory |
| Part A (TMT-A) | Part B (TMT-B) | Test 60-Item | | Verbal Learning |
| | | version (BNT-60) | | Test Retrieval |
| | | | | (RAVLT Retrieval) |
| | | | | Rey Auditory |
| | | | | Verbal Learning |
| | | | | Test Retention |
| | | | | (RAVLT |
| | | | | Recognition) |

Normative data for Z-score calculations were derived from the performance of the participants on each of the cognitive tests adjusted for age, education, sex, and visit. To reduce the effect of cognitively impaired participants on the mean and SD, age-, education-, sex, and visit-adjusted residuals from each domain Z-score model were robustly standardized to have median 0 and robust SD=1, where the robust SD=IQR/1.35, as 1.35 is the IQR (Inter-Quartile Range) of a standard normal distribution.

The participants were then categorized into groups of incident aMCI or early AD (combined into one category a MCI/AD), cognitively normal control (NC), and those who converted to MCI or AD over the course of the study (Converters) based on these composite scores. Impairment was defined as a Z-score 1.35 SD below the cohort median. All participants classified as aMCI met recently revised criteria for the amnestic subtype of MCI. Other behavioral phenotypes of MCI were excluded to concentrate on the amnestic form, which most likely represents nascent Alzheimer's pathology. All early AD participants met recently revised criteria for probable Alzheimer's disease with impairment in memory and at least one other cognitive domain. For the MCI and early AD groups, scores on the measures of memory complaints (MMQ) and activities of daily living (PGC-IADL) were used to corroborate research definitions of these states. All Converters had non-impaired memory at entry to the study (Zₘₑₘ≧4.35), developed memory impairment over the course of the study (Zₘₑₘ≦−1.35) and met criteria for the above definitions of aMCI or AD. To enhance the specificity of the biomarker analyses, NC participants in this study were conservatively defined with Zₘₑₘ±1 SD of the cohort median rather than simply ≧−1.35, and all other Z-scores ≧−1.35 SD.

For each subject, Zₘₑₘ(last), Zₐₜₜ(last), Zₑₓₑ(last), Zₗₐₙ(last), and Zᵥᵢₛ(last) were defined as the age-gender-education-visit-adjusted robust Z-scores for the last available visit for each subject. The aMCI/AD group was defined as those participants whose adjusted Zₘₑₘ was 1 IQR below the median at their last available visit, i.e., Zₘₑₘ(last)≦−1.35. Converters were defined as that subset of the a MCI/AD group whose adjusted Zₘₑₘ at baseline visit 0 was no more than 1 IQR below the median, i.e., Zₘₑₘ(visit=0)>−1.35 and Zₘₑₘ(last)≦−1.35. Participants were classified as NC if they had central scores on all domains at both the first and last visits, i.e., only if they met all of the following six conditions: (i) −1<Zₘₑₘ(last)<1, (ii) −1<Zₘₑₘ(visit=0)<1, (iii) Zₘᵢₙ(last)>−1.35, (iv) Zₘᵢₙ(visit=0)>−1.35, (v) Zₘₐₓ(last)<1.35, and (vi) Zₘₐₓ(visit=0)<1.35, where Zₘₐₓ(last) and Zₘₐₓ(visit=0) denote the maximum of the five adjusted Z-scores at the last and first visits, respectively. Zₘₑₘ for normal participants had to be within 0.74 IQR (1 SD) of the median, rather than just 1 IQR (1.35 SD), to guarantee that they were >0.25 IQR (0.35 SD) from aMCI/AD participants.

After three years of being in the study, (December, 2010), 202 participants had completed a baseline and two yearly visits. At the third visit, 53 participants met criteria for aMCI/AD and 96 met criteria for NC. Of the 53 aMCI/AD participants, 18 were Converters and 35 were incident aMCI or AD. The remaining 53 participants did not meet the criteria for either group and were not considered for biomarker profiling. Some of these individuals met criteria for non-amnestic MCI and many had borderline or even above average memory scores that precluded their inclusion as either aMCI/AD or NC. 53 of the NC participants were matched to the 53 aMCI/AD participants based on sex, age, and education level. Blood samples were obtained on the last available study visit for the 53 MCI/AD and the 53 NC for biomarker discovery. Two blood samples from each of the 18 Converters were also included: one from the baseline visit (Converterₚᵣₑ) when Zₘₑₘ was non-impaired and one from the third visit (Converterₚₒₛₜ) when Zₘₑₘ was impaired and they met criteria for either aMCI or AD. Thus, at total of 124 samples from 106 participants were analyzed.

Internal cross-validation was employed to validate findings from the discovery phase. Blood samples for validation were identified at the end of the fifth year of the study and all 106 participants included in the discovery phase were excluded from consideration for the validation phase. Cognitive composite Z-scores were re-calculated based on the entire sample available and the same procedure and criteria were used to identify samples for the validation phase. A total of 145 participants met criteria for a group: 21aMCI/AD and 124 NC. Of the 21 aMCI/AD, 10 were Converters. 20 of the NC participants were matched to the aMCI/AD participants on the basis of age, sex, and education level as in the discovery phase. In total, 41 participants contributed samples to the validation phase and, as before, the 10 Converters also contributed a baseline sample (Converterₚᵣₑ) for a total of 51 samples.

Neurocognitive Statistical Analyses

The neurocognitive analyses were designed to demonstrate the general equivalence of the discovery and validation samples on clinical and cognitive measures. Separate Multivariate Analysis of Variance (MANOVA's) tests were used to examine discovery/validation group performance on the composite Z-scores and on self-report measures of memory complaints, memory related functional impairment, depressive symptoms, and a global measure of cognitive function. In the first MANOVA, biomarker sample (discovery, validation) was the independent variable and MMQ, IADL, GDS, and MMSE were the dependent variables. In the second MANOVA, biomarker sample (discovery, validation) was the independent variable and the five cognitive domain Z-scores (Zₐₜₜ, Zₑₓₑ, Zₗₐₙ, Zₘₑₘ, and Zᵥᵢₛ) were the dependent variables. Significance was set at alpha=0.05 and Tukey's HSD procedure was used for post-hoc comparisons. All statistical analyses were performed using SPSS (version 21).

### Example 2—Neural Derived Exosomal Analysis

This investigation was performed on a subset of the subjects from the cohort described in a previous study regarding plasma lipidomics. See Mapstone, M., et al. Nat Med 20, 415-418 (2014), which is incorporated by reference. Stored plasma specimens were retrieved for analysis from 37 cognitively unimpaired subjects, including 10 Normal Controls (NC) and 27 Converterₚᵣₑ individuals. See definitions of clinical groups in World Health Organization. Dementia: a public health priority, (World Health Organization, Geneva, 2012. ISBN 978-92-4-156445-8), which is incorporated by reference. In addition, specimens were obtained from 10 subjects entering the study with evidence of aMCI or AD, and 27 matched specimens from the Converterₚᵣₑ individuals that phenoconverted to Converterₚₒₛₜ. Demographic data summarizing our study subjects is provided in Table 4.

TABLE 4
Subject Demographics

| Diagnostic | | | | |
|---|---|---|---|---|
| Group | NC | Converterₚᵣₑ | Converterₚₒₛₜ | aMCI/AD |
| Number of | 10 | 27 | 27 | 10 |
| Subjects | | | | |
| Age (yrs. ± std. | 79.2 ± 4.0 | 80.1 ± 4.1^{b} | 82.2 ± 4.0^{a,b} | 81.3 ± 4.9 |
| dev.) | | | | |
| Gender | 60 | 56 | 56 | 50 |
| (% Female) | | | | |
| Education | 14.6 | 15.1 | 15.1 | 15.7 |
| (yrs.) | | | | |
| MMSE | 28.5 ± 1.5 | 28.6 ± 2.5^{d} | 27.0 ± 2.0^{c,d} | 25.0 ± 3.7^{c} |
| (±std. dev.) | | | | |
| APOE allele | 40/20 | 19/0 | 19/0 | 50/20 |
| (% e4/e2) | | | | |

In the table above, aMCI/AD=cognitive impairment at study entry; APOE=apolipoprotein E gene; Converterₚᵣₑ=cognitively unimpaired, prior to phenoconversion; Converterₚₒₛₜ=phenoconverted to cognitively impaired; M/F=male/female; MMSE=mini mental status examination, NC=individuals at study entry found to have normal cognition and maintain it throughout the study; std. dev.=standard deviation; yrs.=years. All statistical comparisons between groups were performed via two-tailed t-tests and were not significant, except: a approaches statistical significance compared with NC, p=0.05 for independent samples; b significant difference between paired groups, p<0.001; c significant difference from NC, p<0.05 for independent samples; d Significant difference between paired groups, p<0.01

Isolation of Exosomes from Plasma for ELISA Quantification of Exosome Proteins

One-half ml of plasma was incubated with 0.15 ml of thromboplastin-D (Fisher Scientific, Inc., Hanover Park, Ill.) at room temperature for 60 min, followed by addition of 0.35 ml of calcium- and magnesium-free Dulbecco's balanced salt solution (DBS⁻²) with protease inhibitor cocktail (Roche Applied Sciences, Inc., Indianapolis, Ind.) and phosphatase inhibitor cocktail (Pierce Halt, Thermo Scientific, Inc., Rockford, Ill.). After centrifugation at 1,500×g for 20 min, supernates were mixed with 252 μl of ExoQuick™ exosome precipitation solution (EXOQ; System Biosciences, Inc., Mountainview, Calif.), and incubated for 1 hr at 4° C. Resultant exosome suspensions were centrifuged at 1,500×g for 30 min at 4° C. and each pellet was re-suspended in 250 μl of DBS⁻² with inhibitor cocktails before immunochemical enrichment of exosomes from a neural source, as described for immune cell exosomes in Mitsuhashi, M., et al. FASEB J. 27, 5141-5150 (2013), which is incorporated by reference.

Each sample was incubated sequentially for 1 hr at 4° C. with 2 μg of mouse anti-human NCAM antibody (ERIC 1, sc-106, Santa Cruz Biotechnology, Santa Cruz, Calif.), that had been biotinylated with the EZ-Link sulfo-NHS-biotin system (Thermo Scientific, Inc.), and then 25 μl of streptavidin-agarose resin (Thermo Scientific, Inc.). After centrifugation at 200×g for 10 min at 4° C., each pellet was resuspended in 0.5 ml of DBS⁻² with 2 g/100 ml of BSA, 0.10% Tween 20 and the inhibitor cocktails by incubation for 30 min at 37° C. with vortex-mixing and was stored at −80° C. prior to ELISAs. Relative yields of exosomes from plasma at this stage were compared using sources from all subject groups. The respective mean levels of tau and Aβ₁₋₄₂ species, along with CD81 extracted from exosomes, allowed normalization of each protein analyte to the exosome marker CD81, as previously reported in Fiandaca, M., et al. Alzheimers Dement (In Review) (2014) and Mitsuhashi, M., et al. FASEB J. 27, 5141-5150 (2013), which are incorporated by reference.

Exosome proteins were quantified by ELISA kits for human amyloid beta isoform 1-42 (Aβ₁₋₄₂), human Total tau (T-tau) and human phosphorylated-5396-tau (p-tau-s396) (Life Technologies/Invitrogen, Camarillo, Calif.), human phosphorylated-T181-tau (p-tau-t181) (Innogenetics Division of Fujirebio US, Inc., Alpharetta, Ga.) and human CD81 (Holzel Diagnostika-Cusabio, Cologne, Germany), with verification of the CD81 antigen standard curve using human purified recombinant CD81 antigen (Origene Technologies, Inc., Rockville, Md.), as previously described, and according to suppliers' directions. The mean value for all determinations of CD81 in each assay group was set at 1.00 and the relative values for each sample used to normalize their recovery.

Statistical Analyses

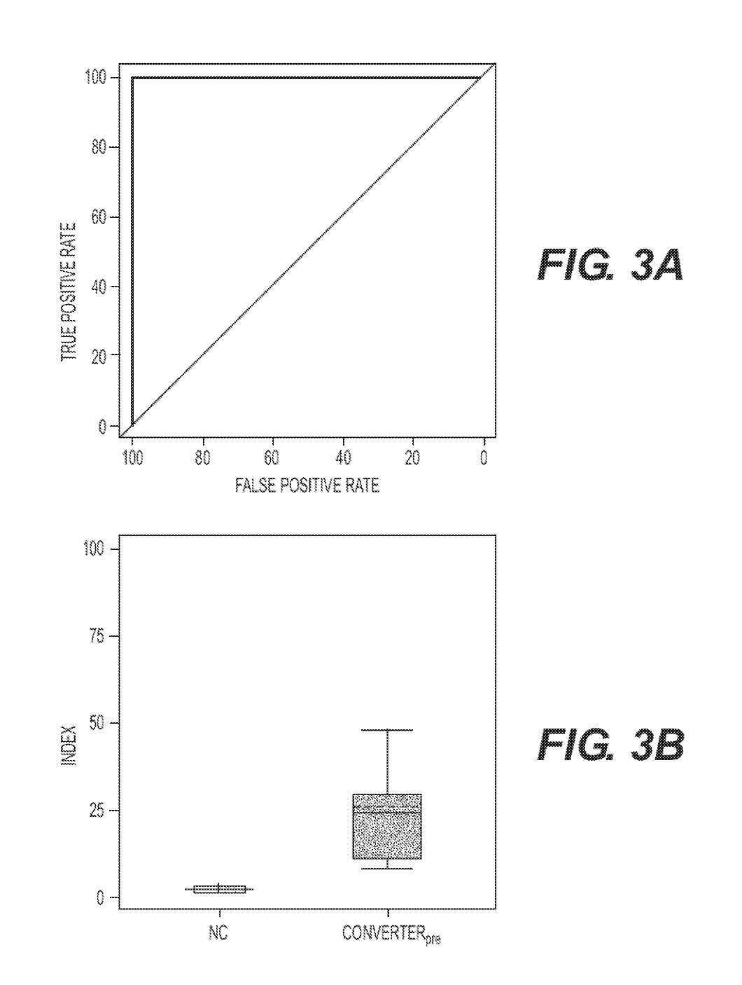

Data analysis was performed using IBM SPSS Statistics version 22 for Mac (64 bit edition), including parametric and nonparametric testing, as defined specifically for each result. ROC curves were constructed with the R package ‘pROC’ to examine the diagnostic value of each analyte. The AUCs of the ROC curves and their 95% confidence intervals (CIs) were evaluated as measures of diagnostic accuracy. A multivariate logistic regression analysis was performed to identify the combination of those analytes that yielded optimal separation between NCs and Converterₚᵣₑ. Because there are significant differences in the concentration level of all four analytes, between NCs and Converterₚᵣₑ, the combined classifier will yield a complete separation of the two groups. Given that complete separation would imply the nonexistence of the regular estimator, the hidden logistic regression model with Maximum Estimated Likelihood (MEL) estimator was employed, (described in Rousseeuw, P. J. & Christmann, A. Computational Statistics & Data Analysis 43, 315-332 (2003), which is incorporated by reference), for analysis with the R package ‘hlr’. The MEL method is an extension of the classical logistic regression model, where it assumes that the true response cannot be observed, but that there exists an observable variable which is strongly related to the true response. The final logistic regression model can be written as logit π=constant+aX₁+bX₂+cX₃+dX₄, where π is probability of a patient belonging to the Converterₚᵣₑ group, X₁, X₂, X₃, X₄ representing the four analytes, and a, b, c, d are the coefficients of the regression equation. The combined classifier yields a complete separation of the two groups with AUC=1 (FIG. 3a). With this classifier, an index was also defined that will be useful for classification of new patients into those with high or medium risk of phenoconversion and with low risk, as index is defined as (logit π+20)/2. With this index, a clear separation of NCs and Converterₚᵣₑ can be shown. (FIG. 3b). The variance of the index for the Converterₚᵣₑ group is fairly large compared to NCs. This is due to the fact that the variance of the concentration level of the four analytes for the Converterₚᵣₑ group are also much larger compared to NCs.

Results

Plasma samples from a recently reported longitudinal study cohort were analyzed (Mapstone, M., et al. Nat Med 20, 415-418 (2014)). The samples were from cognitively normal controls (NC), cognitively normal subjects that later phenoconverted (Converterₚᵣₑ) to amnestic mild cognitive impairment (aMCI) or AD, samples from Converterₚᵣₑ individuals after phenoconversion (to Converterₚₒₛₜ), and samples from subjects with either aMCI or AD (aMCI/AD). The subject samples (Table 4) were matched for age, gender, education, MMSE and APOE allele status. Group comparisons were not significant except as detailed herein. Detailed neuropsychological assessments for this cohort, as described previously (Mapstone, M., et al. Nat Med 20, 415-418 (2014)), disclosed no significant cognitive difference between NC and Converterₚᵣₑ, or Converterₚₒₛₜ and aMCI/AD groups. Significant differences were observed between NC and Converterₚₒₛₜ, NC and aMCI/AD, and Converterₚᵣₑ and Converterₚₒₛₜ groups in the original neuropsychological results (Mapstone, M., et al. Nat Med 20, 415-418 (2014)) and the MMSE data, consistent with their diagnostic categories. APOE allele frequency did not differ among groups.

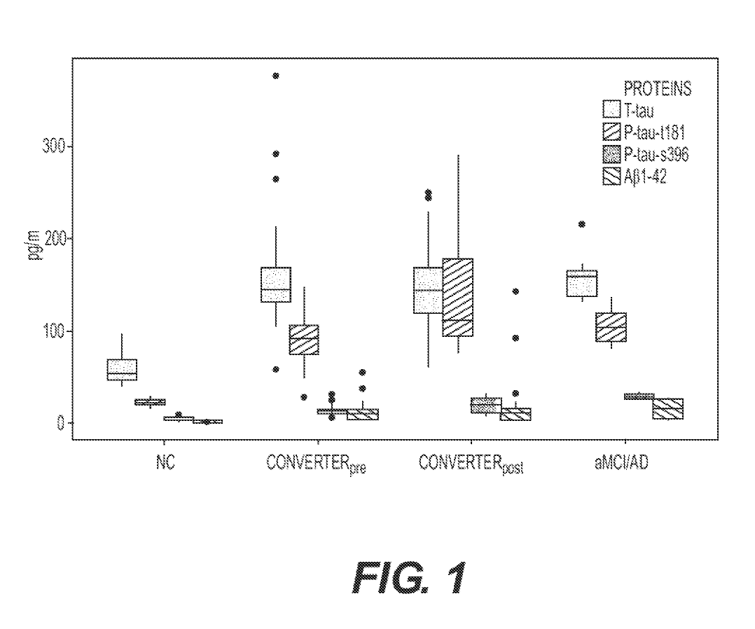

It was reasoned that exosomes of presumptive nervous system origin could convey cargos including proteins of pathogenic relevance to dementia. NCAM positive plasma exosomes were immuno-isolated and analyzed for their protein cargo by ELISA. The exosomes were interrogated for cargo proteins know to be dysregulated in AD: total tau (T-tau), phosphorylated-tau species, at tyrosine 181 (P-tau-t181), and serine 396 (P-tau-s396), and Aβ₁₋₄₂, for each of the clinical groups (Table 5 and FIG. 1). Statistical analyses for between-group comparisons were performed using the Student's unpaired t-test. Given the relatively small sample size, a Kolmogorov-Smirnov normality test was used to determine whether plasma concentrations were normally distributed. When the concentrations did not consistently satisfy this test for normality, the Wilcoxon signed-rank test was also used to compare plasma concentrations between the groups. The nonparametric analysis results support the parametric t-test results.

TABLE 5
Neural-derived Plasma Exosome Protein Cargo Levels

| Diagnostic | | | | |
|---|---|---|---|---|
| Group | NC | Converterₚᵣₑ | Converterₚₒₛₜ | aMCI/AD |
| Number of | 10 | 27 | 27 | 10 |
| specimens | | | | |
| T-tau | 59.2 (5.5) | 163.3 (12.3) | 150.7 (8.7) | 157.5 (7.9) |
| pg/ml | | [275] | [255] | [266] |
| (±SEM) | | | | |
| [% increase | | | | |
| from NC] | | | | |
| P-tau-t181 | 22.3 (1.0) | 91.0 (4.9) | 136.6 (12.1) | 105.1 (6.2) |
| pg/ml | | [408] | [613] | [471] |
| (±SEM) | | | | |
| [% increase | | | | |
| from NC] | | | | |
| P-tau-s396 | 4.6 (0.6) | 12.5 (0.8) | 18.6 (1.6) | 28.72 (0.843) |
| pg/ml | | [272] | [404] | [624] |
| (±SEM) | | | | |
| [% increase | | | | |
| from NC] | | | | |
| Aβ(1-42) | 0.84 (0.09) | 12.57 (2.21) | 17.92 (5.80) | 15.00 (3.24) |
| pg/ml | | [1496] | [2133] | [1786] |
| (±SEM) | | | | |
| [% increase | | | | |
| from NC] | | | | |
| T-tau/Aβ | 70.5 | 13.0 | 8.4 | 10.5 |
| (1-42) | | | | |
| Ratio | | | | |

In the Table above, Aβ(1-42)=amyloid β fragment (1-42); aMCI/AD=amnestic mild cognitive impairment or Alzheimer's disease at study entry; Converterₚᵣₑ=cognitively unimpaired, prior to phenoconversion to aMCI or AD; Converterₚₒₛₜ=cognitively impaired, after phenoconversion to aMCI or AD; NC=normal cognition at study entry and maintained throughout study; P-tau-t181=phosphorylated tau at tyrosine 181; P-tau-s396=phosphorylated tau at serine 396; SEM=standard error of the mean; T-tau=Total tau is the combined value of all measureable tau species. Data rows 2 through 5 report the average concentration of the four analytes in the four clinical groups, with standard errors appearing in parentheses and % increase in the protein levels compared to NC. Each of the three clinical groups showed significant differences compared to NC (p<0.001) with all four exosomal protein levels.

All measured exosomal cargo protein levels (FIG. 1, Table 5) were significantly elevated in the Converterₚᵣₑ, Converterₚₒₛₜ, and aMCI/AD groups compared to the NC group. Each of the three non-NC groups showed significant differences from NC (p<0.001) for all four protein levels. Pairwise comparisons also showed that P-tau-t181 was significantly different between Converterₚᵣₑ and Converterₚₒₛₜ (p<0.01). In addition, significant differences in P-tau-s396 were also noted between Converterₚᵣₑ and Converterₚₒₛₜ (p<0.001), Converterₚᵣₑ and aMCI/AD (p<0.001), and Converterₚₒₛₜ and aMCI/AD (p<0.001). T-tau percentage increase compared to NC levels remained relatively stable (^{˜}250%) from Converterₚᵣₑ to Converterₚₒₛₜ to aMCI/AD.

T-tau levels were noted to be increased by ^{˜}250% from NC levels in the other three clinical groups, without much difference in this protein level noted between preclinical and clinical stages of disease.

In contrast, the P-tau-t181 levels were >400% higher in the Converterₚᵣₑ group compared to NC, and this increase peaked at >600% of NC levels following the transition to Converterₚₒₛₜ before dropping back to ^{˜}470% of NC levels in the aMCI/AD group. Pairwise protein level comparisons also showed that P-tau-t181 was also significantly different between Converterₚᵣₑ and Converterₚₒₛₜ (p<0.01).

Significant pairwise differences were noted in P-tau-s396 between Converterₚᵣₑ and Converterₚₒₛₜ (p<0.001), Converterₚᵣₑ and aMCI/AD (p<0.001), and Converterₚₒₛₜ and aMCI/AD (p<0.001). P-tau-s396 showed progressively increasing levels with stage of disease, compared to NC. Asymptomatic Converterₚᵣₑ subjects had levels ^{˜}270% higher than NC, while early clinical disease (Converterₚₒₛₜ) and later disease (aMCI/AD) showed progressively increasing levels, ^{˜}400% and >600% greater than NC, respectively.

Finally, Aβ₁₋₄₂ was most significantly increased compared to NC, in both the preclinical AD group (Converterₚᵣₑ) as well as following phenoconversion to manifest disease (Converterₚₒₛₜ and aMCI/AD). The Aβ₁₋₄₂ group actually showed the greatest percentage increase from NC levels in the other clinical groups. An elevation by 1500% was noted between Converterₚᵣₑ and NC, while this increased to >2100% of NC levels in Converterₚₒₛₜ, and finally dropped to nearly 1800% of NC levels in aMCI/AD.

A significant reduction in the T-tau/Aβ₁₋₄₂ ratio is evident between NC and the other groups. The largest pairwise difference was noted between NC and the other three individual clinical groups. No significant differences were noted in comparing this ratio between the three non-NC groups, with the ratio remaining ^{˜}10 from preclinical to clinical disease. The dramatic change in T-tau/Aβ₁₋₄₂ ratio noted between the two asymptomatic groups (NC and Converterₚᵣₑ) was likely primarily due to the significant elevation of Aβ₁₋₄₂ levels within the plasma exosomes, noted with disease progression compared to NC, and contrary to consensus opinion regarding Aβ1₋₄₂ levels in plasma, as reported in Rissman, R., et al., J Neural Transm 119, 843-850 (2012) or CSF as reported in Craig-Schapiro, R., et al., Neurobiol Dis. 35, 128-140 (2009) and Buchhave, P., et al. Archives of General Psychiatry 69, 98-106 (2012).

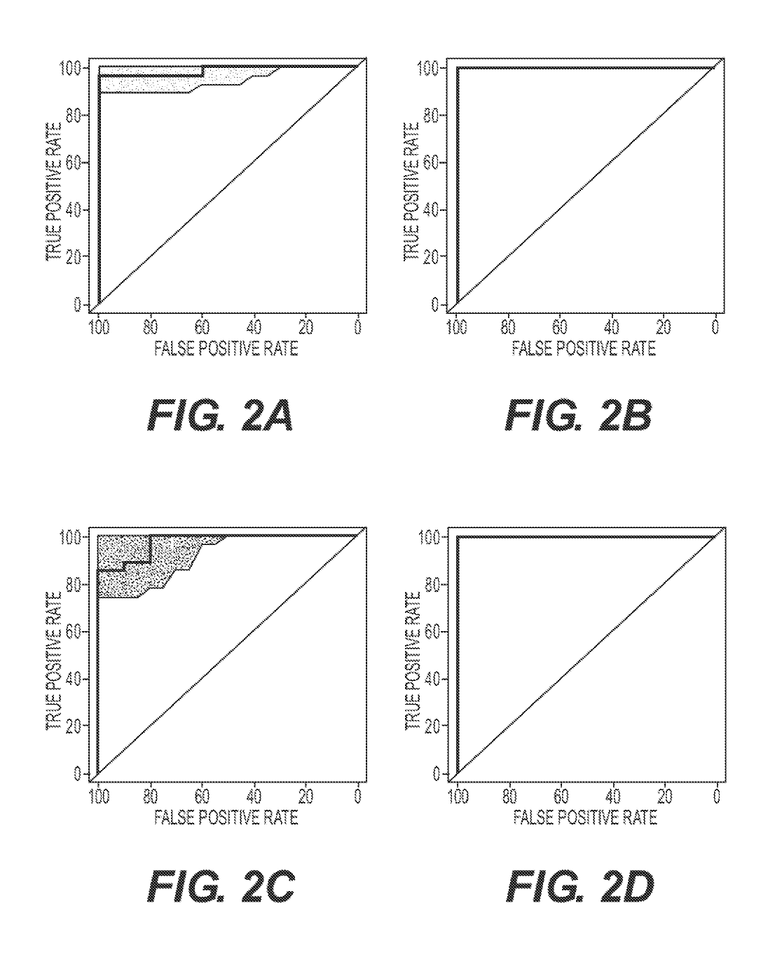

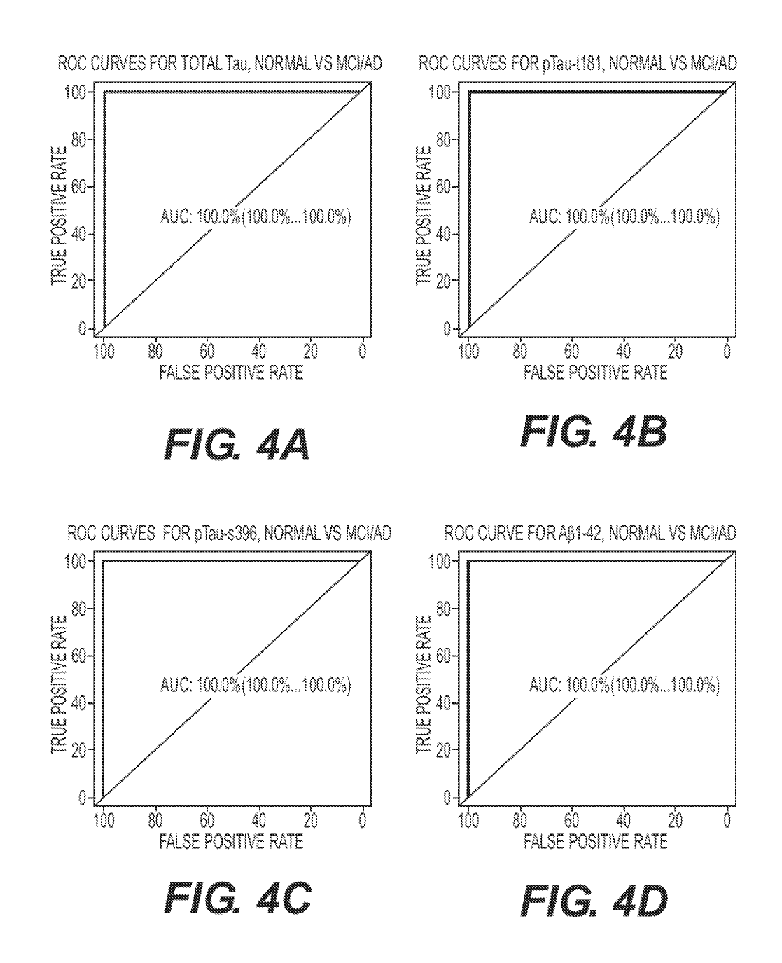

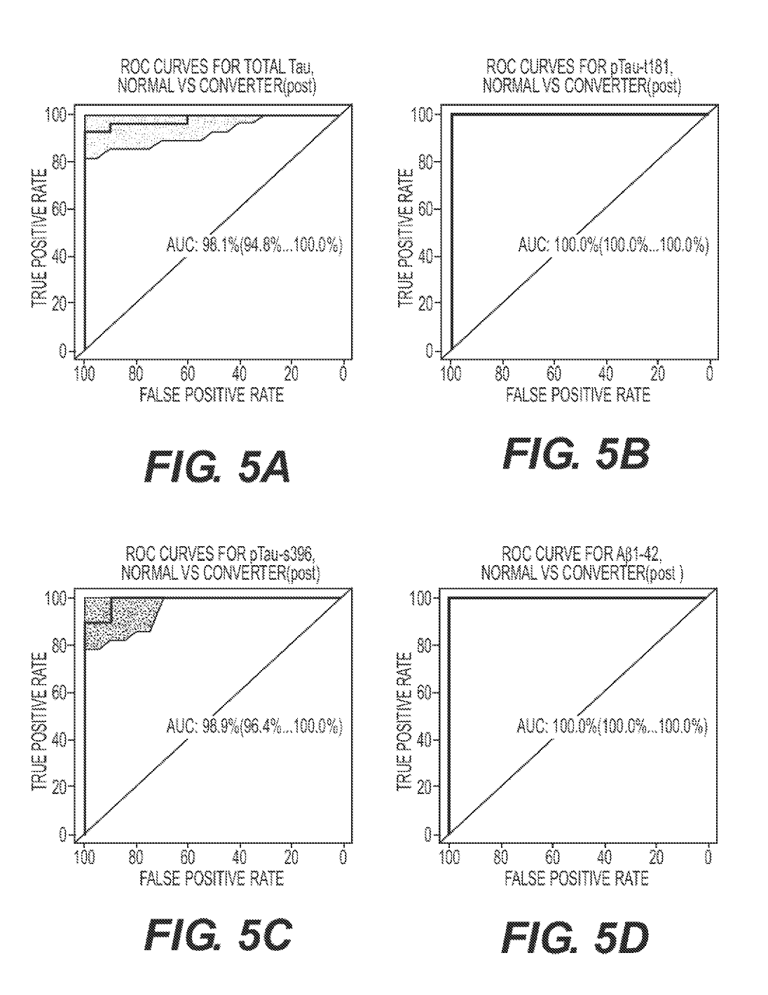

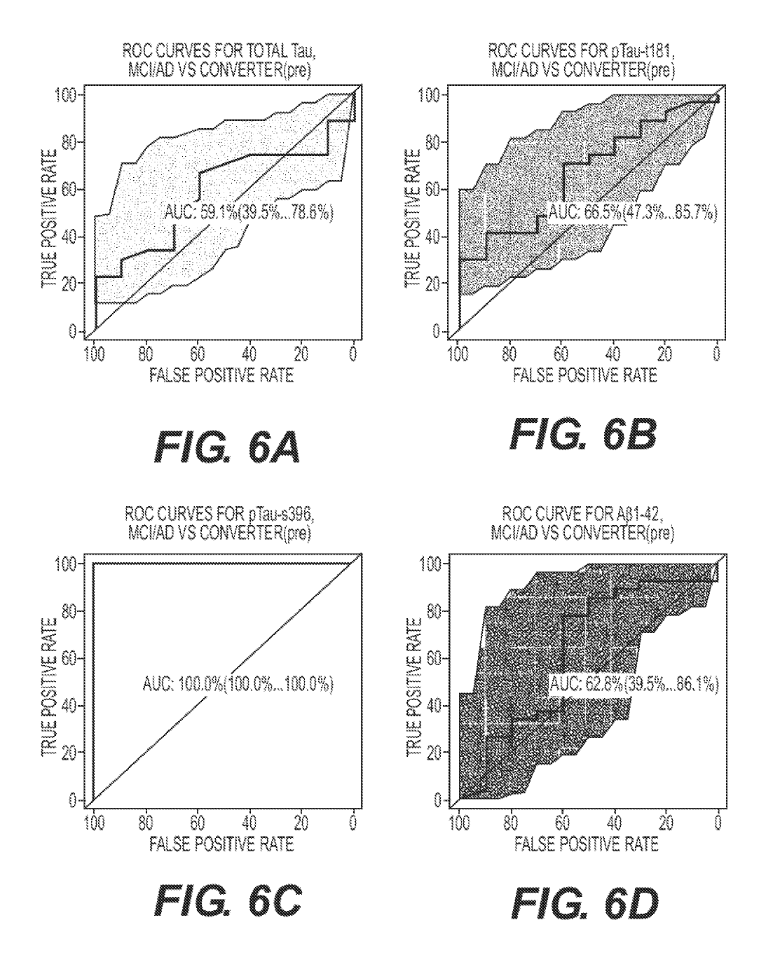

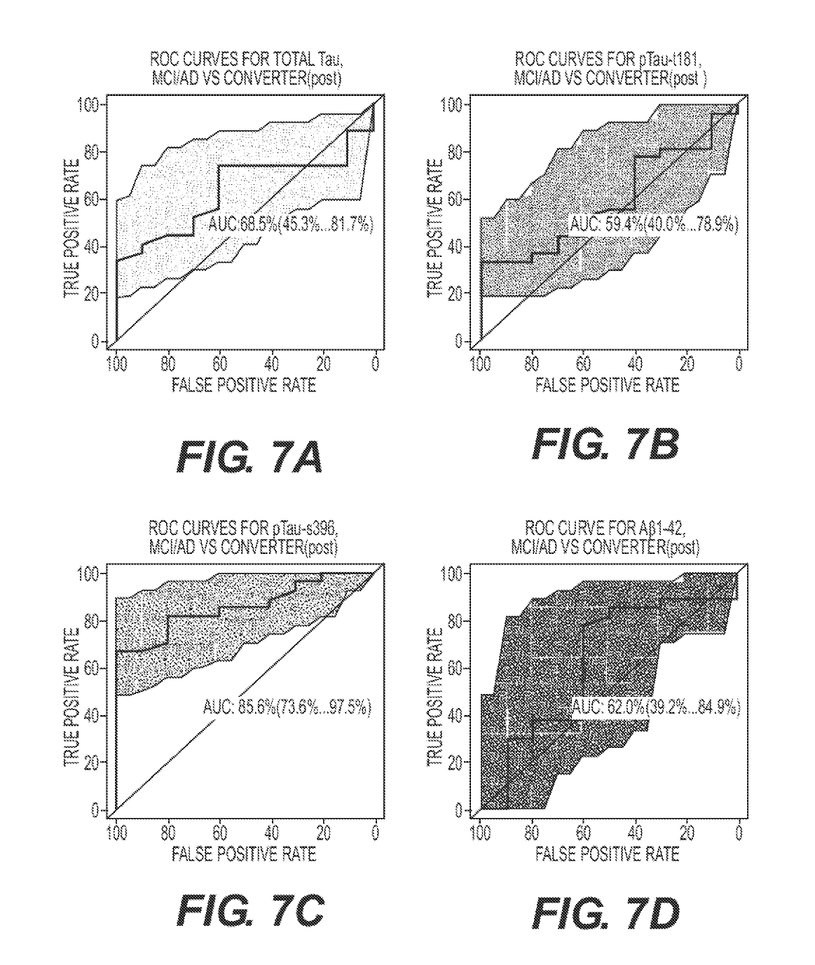

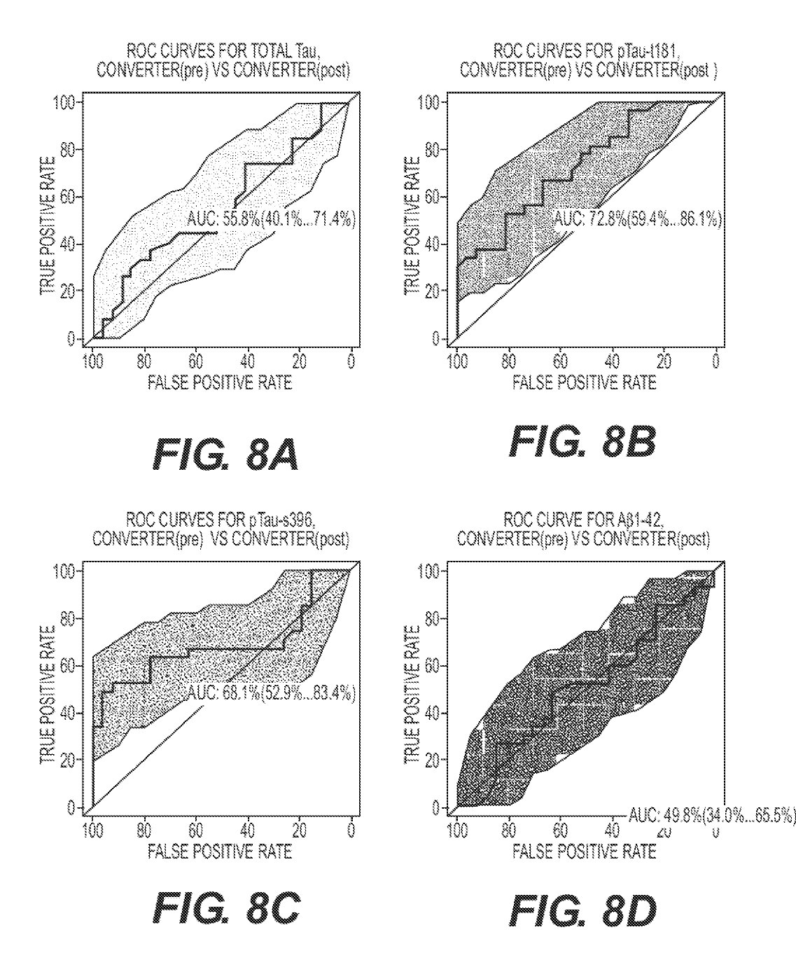

Receiver operating characteristic (ROC) analyses was performed using each of the four protein analytes (T-tau, P-tau-t181, P-tau-s396, or Aβ₁₋₄₂) independently (See FIGS. 4-8). Of particular interest, each protein analyte differentiated the two cognitively unimpaired groups (NC and Converterₚᵣₑ) with highly significant accuracy. The ROC area under the curves (AUCs) values featured 0.985 for T-tau, 1.00 for P-tau-t181, 0.974 for P-tau-s396, and 1.00 for Aβ₁₋₄₂ (FIG. 2 a-d). In addition, the combined classifier using all four analytes yields an ROC AUC of 1.00 (FIG. 3a), and defines a Plasma Exosome Index (PEI) that allows accurate predictive capabilities for individually determined values (FIG. 3b) based on the absence of overlap between NC and clinical (at risk) groups.

Of the 35 million Americans greater than age 65, approximately 60% are women and 40% are men. Based on recent estimates, the prevalence of AD in women and men age 71 and older is 16% and 11%, respectively. The latter prevalence estimates of AD in women and men, therefore, provide a positive predictive value (PPV) in cognitively normal individuals, based on the previous study involving a 10 lipid panel results (Mapstone, M., et al. Nat Med 20, 415-418 (2014)), of 63.1% for women, and 52.7% for men, and the negative predictive value (NPV) being 98% for women and 98.6% for men. Using the reported exosome findings, the PPV and NPV results, using the same prevalence estimates, achieve 100% for both women and men using the combined classifier for the four analytes (FIG. 3a), and the calculated Plasma Exosome Index value (FIG. 3b).

Thus NCAM positive exosomal cargos are useful in predicting phenoconversion to aMCI or AD. CNS derived exosomes may constitute a new neuroendocrine-like central to peripheral signaling mechanism which requires further elucidation. In addition, highly accurate predictive biosignatures of manifest AD enable an era for new secondary prevention clinical trials.

### Example 3—Lipidopmic Analysis

Lipidomics Methods

LC/MS-grade acetonitrile (ACN), Isopropanol (IPA), water and methanol were purchased from Fisher Scientific (New Jersey, USA). High purity formic acid (99%) was purchased from Thermo-Scientific (Rockford, Ill.). Debrisoquine, 4-Nitrobenzoic acid (4-NBA), Pro-Asn, Glycoursodeoxycholic acid, Malic acid, were purchased from Sigma (St. Louis, Mo., USA). All lipid standards including 14:0 LPA, 17:0 Ceramide, 12:0 LPC, 18:0 Lyso PI and PC(22:6/0:0) were procured from Avanti Polar Lipids Inc. (USA).

Lipid Extraction

Briefly, the plasma samples were thawed on ice and vortexed. For lipid extraction, 25 μL of plasma sample was mixed with 175 μL of extraction buffer (25% acetonitrile in 40% methanol and 35% water) containing internal standards [10 μL of debrisoquine (1 mg/mL), 50 μL of 4, nitro-benzoic acid (1 mg/mL), 27.3 μl of Ceramide (1 mg/mL) and 2.5 μL of LPA (lysophosphatidic acid) (4 mg/mL) in 10 mL). The samples were incubated on ice for 10 minutes and centrifuged at 14,000 rpm at 4° C. for 20 minutes. The supernatant was transferred to a fresh tube and dried under vacuum. The dried samples were reconstituted in 200 μL of buffer containing 5% methanol, 1% acetonitrile and 94% water. The samples were centrifuged at 13,000 rpm for 20 minutes at 4° C. to remove fine particulates. The supernatant was transferred to a glass vial for UPLC-ESI-Q-TOF-MS analysis.

UPLC-ESI-QTOF-MS Based Data Acquisition for Untargeted Lipidomic Profiling

Each sample (2 μL) was injected onto a reverse-phase CSH C18 1.7 μM 2.1×100 mm column using an Acquity H-class UPLC system (Waters Corporation, USA). The gradient mobile phase comprised of water containing 0.1% formic acid solution (Solvent A), 100% acetonitrile (Solvent B) and 10% acetonitrile in isopropanol (IPA) containing 0.1% formic acid and 10 mM Ammonium formate (Solvent C). Each sample was resolved for 13 minutes at a flow rate of 0.5 mL/min for 8 min and then 0.4 mL/min from 8 to 13 min. The UPLC gradient consisted of 98% A and 2% B for 0.5 min then a ramp of curve 6 to 60% B and 40% A from 0.5 min to 4.0 min, followed by a ramp of curve 6 to 98% B and 2% A from 4.0 to 8.0 min, then ramped to 5% B and 95% C from 9.0 min to 10.0 min at a flow rate of 0.4 ml/min, and finally to 98% A and 2% B from 11.0 min to 13 minutes. The column eluent was introduced directly into the mass spectrometer by electrospray ionization. Mass spectrometry was performed on a Quadrupole-Time of Flight (Q-TOF) instrument (Xevo G2 QTOF, Waters Corporation, USA) operating in either negative (ESI⁻) or positive (ESI⁺) electrospray ionization mode with a capillary voltage of 3200 V in positive mode and 2800 V in negative mode, and a sampling cone voltage of 30 V in both modes. The desolvation gas flow was set to 750 L h⁻¹ and the temperature was set to 350° C. while the source temperature was set at 120° C. Accurate mass was maintained by introduction of a lock spray interface of leucine—enkephalin (556.2771 [M+H]⁺ or 554.2615 [M−H]⁻) at a concentration of 2 pg/μl in 50% aqueous acetonitrile and a rate of 2 μl/min. Data were acquired in centroid MS mode from 50 to 1200 m/z mass range for TOF-MS scanning as single injection per sample and the batch acquisition was repeated to check experimental reproducibility. For the metabolomics profiling experiments, pooled quality control (QC) samples (generated by taking an equal aliquot of all the samples included in the experiment) were run at the beginning of the sample queue for column conditioning and every ten injections thereafter to assess inconsistencies that are particularly evident in large batch acquisitions in terms of retention time drifts and variation in ion intensity over time. This approach has been recommended and used as a standard practice by leading metabolomics researchers. A test mix of standard lipds was run at the beginning and at the end of the run to evaluate instrument performance with respect to sensitivity and mass accuracy. The overlay of the total ion chromatograms of the quality control samples depicted excellent retention time reproducibility. The sample queue was randomized to remove bias. The TICs for each of the three groups showed characteristic patterns.

Stable Isotope Dilution—Multiple Reaction Monitoring Mass Spectrometry (SID-MRM-MS)

Targeted metabolomic analysis of plasma sample was performed using the Biocrates Absolute-IDQ P180 (BIOCRATES, Life Science AG, Innsbruck, Austria). This validated targeted assay allows for simultaneous detection and quantification of lipids in plasma samples (10 μL) in a high throughput manner. The methods have been described in detail. The plasma samples were processed as per the instructions by the manufacturer and analyzed on a triple quadrupole mass spectrometer (Xevo TQ-S, Waters Corporation, USA) operating in the MRM mode. The measurements were made in a 96 well format for a total of 148 samples, seven calibration standards and three quality control samples were integrated in the kit.

Briefly, the flow injection analysis (FIA) tandem mass spectrometry (MS/MS) method was used to quantify a panel of 144 lipids simultaneously by multiple reaction monitoring. Absolute quantification was achieved by extrapolating from a standard curve. The other lipds were resolved on the UPLC and quantified using scheduled MRMs. The kit facilitated absolute quantitation of 21 amino acids, hexose, carnitine, 39 acylcarnitines, 15 sphingomyelins, 90 phosphatidylcholines and 19 biogenic amines. Data analysis was performed using the MetIQ software (Biocrates) while the statistical analyses were performed using the STAT pack module v3 (Biocrates). The abundance was calculated from area under the curve by normalizing to the respective isotope labeled internal standard. The concentration is expressed as nmol/L. Quality control samples were used to assess reproducibility of the assay. The mean of the coefficient of variation (CV) for the 180 lipids was 0.08 and 95% of the lipids had a CV of <0.15.

Lipidomics Statistical Analyses

The m/z features of lipids were normalized with log transformation that stabilized the variance followed with a quantile normalization to make the empirical distribution of intensities the same across samples. The lipds were selected among all those known to be identifiable using a ROC regularized learning technique, based on the least absolute shrinkage and selection operator (LASSO) penalty as implemented with the R package ‘glmnet’, which uses cyclical coordinate descent in a pathwise fashion. The regularization path over a grid of values was obtained for the tuning parameter lambda through 10-fold cross-validation. The optimal value of the tuning parameter lambda, which was obtained by the cross-validation procedure, was then used to fit the model. All the features with non-zero coefficients were retained for subsequent analysis. The classification performance of the selected lipids was assessed using area under the ROC (receiver operating characteristic) curve (AUC). The ROC can be understood as a plot of the probability of classifying correctly the positive samples against the rate of incorrectly classifying true negative samples. Thus the AUC measure of an ROC plot is actually a measure of predictive accuracy. To maintain rigor of independent validation, the simple logistic model with the ten lipid panel was used, although a more refined model can yield greater AUC.

All references disclosed herein are expressly incorporated by reference.

What is claimed is:

1. A method of determining if a subject has an increased risk of suffering from memory impairment, the method comprising
a) analyzing at least one sample from the subject to determine the subject's exosomal profile, and
b) comparing the value of the subject's exosomal profile with the value obtained from subjects determined to define a normal exosomal profile, to determine if the subject's exosomal profile is altered compared to a normal exosomal profile,
wherein a change in the value of the subject's exosomal profile is indicative that the subject has an increased risk of suffering from future memory impairment compared to those defined as having a normal exosomal profile.

2. The method of claim 1, wherein the exosomal profile comprises neurally-derived exosomes profile taken from the subject's blood

3. The method of claim 2, wherein the exosomes are NCAM-positive.

4. The method of claim 3, wherein the NCAM-positive exosomes comprise one or more proteins or fragments thereof that are derived from nervous system tissue.

5. The method of claim 4, wherein the one or more proteins or fragments thereof are selected from the group consisting of Total tau protein, phosphorylated tau-T181 protein, phosphorylated tau-S396 protein and amyloid β₁₋₄₂.

6. The method of claim 5, wherein the exosomes comprise at least two, three or four proteins or fragments thereof selected from the group consisting of Total tau protein, phosphorylated tau-T181 protein, phosphorylated tau-S396 protein and amyloid β₁₋₄₂.

7. A method of monitoring the progression of memory impairment in a subject, the method comprising
a) analyzing at least two blood samples from the subject with each sample taken at different time points to determine the values of each of the subject's exosomal profiles, and
b) comparing the values of the subject's exosomal profiles over time to determine if the subject's exosomal profile is changing over time,
wherein a change in the subject's exosomal value over time is indicative that the subject's risk of suffering from memory impairment is increasing over time.

8. The method of claim 7, wherein the exosomal profile comprises neurally-derived exosomes taken from the subject's blood.

9. The method of claim 8, wherein the exosomes are NCAM-positive.

10. The method of claim 9, wherein the NCAM-positive exosomes comprise one or more proteins or fragments thereof that are derived from nervous system tissue.

11. The method of claim 10, wherein the one or more proteins or fragments thereof are selected from the group consisting of Total tau protein, phosphorylated tau-T181 protein, phosphorylated tau-S396 protein and amyloid β₁₋₄₂.

12. The method of claim 11, wherein the exosomes comprise at least two, three or four proteins or fragments thereof selected from the group consisting of Total tau protein, phosphorylated tau P-T181 protein, phosphorylated tau P-S396 protein and amyloid β₁₋₄₂.

13. A method of monitoring the progression of a treatment for memory impairment in a subject, the method comprising
a) analyzing at least two samples from a subject undergoing treatment for memory impairment with each sample taken at different time points to determine the values of each of the subject's exosomal profiles, and
b) comparing the values of the subject's exosomal profiles over time to determine if the subject's exosomal profile is changing over time in response to the treatment,
wherein a lack of change or a further deviation from a normal exosomal profile in the subject's exosomal profile is indicative that the treatment for memory impairment is not effective, and wherein an approximation of the subject's exosomal profile over time towards a normal exosomal profile is indicative that the treatment for memory impairment is effective in treating memory impairment in the subject.

14. The method of claim 13, wherein the exosomal profile comprises neurally-derived exosomes taken from the subject's blood.

15. The method of claim 14, wherein the exosomes are NCAM-positive.

16. The method of claim 15, wherein the NCAM-positive exosomes comprise one or more proteins or fragments thereof that are derived from nervous system tissue.

17. The method of claim 16, wherein the one or more proteins or fragments thereof are selected from the group consisting of Total tau protein, phosphorylated tau P-T181 protein, phosphorylated tau P-S396 protein and amyloid β₁₋₄₂.

18. The method of claim 17, wherein the exosomes comprise at least two, three or four proteins or fragments thereof selected from the group consisting of Total tau protein, phosphorylated tau P-T181 protein, phosphorylated tau P-S396 protein and amyloid β₁₋₄₂.

19. A method of determining if a subject has an increased risk of suffering from memory impairment, the method comprising
a) analyzing at least one sample from the subject to determine the subject's combined biomarker profile, wherein the combined biomarker profile comprises at least one exosomal constituent and at least one lipid constituent, and
b) comparing the value of the subject's combined biomarker profile with the value obtained from subjects determined to define a normal combined biomarker profile, to determine if the subject's combined biomarker profile is altered compared to a normal combined biomarker profile,
wherein a change in the value of the subject's combined biomarker profile is indicative that the subject has an increased risk of suffering from future memory impairment compared to those defined as having a normal combined biomarker profile.

20. The method of claim 19, wherein the exosomal constituent is isolated from neurally-derived exosomes profile taken from the subject's blood

21. The method of claim 20, wherein the exosomes are NCAM-positive.

22. The method of claim 21, wherein the NCAM-positive exosomes comprise one or more proteins or fragments thereof that are derived from nervous system tissue.

23. The method of claim 22, wherein the one or more proteins or fragments thereof are selected from the group consisting of Total tau protein, phosphorylated tau-T181 protein, phosphorylated tau-S396 protein and amyloid β₁₋₄₂.

24. The method of claim 23, wherein the exosomes comprise at least two, three or four proteins or fragments thereof selected from the group consisting of Total tau protein, phosphorylated tau-T181 protein, phosphorylated tau-S396 protein and amyloid β₁₋₄₂.

25. The method of claim 24, wherein the at least one lipid constituent is a lipid selected from the group consisting of acylcarnitines (ACs) or phosphatidyl cholines (PCs).

26. The method of claim 25, wherein the lipid constituents comprises at least two lipids selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.

27. The method of claim 26, wherein the lipid constituents comprises at least at least three, four, five, six, seven, eight, nine or 10 lipids selected from the group consisting of propionyl AC, lyso PC a C18:2, PC aa C36:6, C16:1-OH, PC aa C38:0, PC aa 36:6, PC aa C40:1, PC aa C40:2, PC aa C40:6 and PC ae C40:6.
